SEQUENCE LISTING (6 sequences)

SEQ ID NO: 1 - DNA, 1725 bp, Respiratory syncytial virus
  nucleotide sequence of F0 from RSV strain A2
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgccggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 2 - protein, 574 aa, Respiratory syncytial virus
  amino acid sequence of F0 from RSV strain A2
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 3 - DNA, 273 bp, Respiratory syncytial virus
  nucleotide sequence of M2-2 from RSV strain A2
atgaccatgccaaaaataatgatactacctgacaaatatccttgtagtataacttccatactaataacaagtagatgtagagttactatgtataatcaaaagaacacactatatttcaaccaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataa

SEQ ID NO: 4 - protein, 90 aa, Respiratory syncytial virus
  amino acid sequence of M2-2 from RSV strain A2
MTMPKIMILPDKYPCSITSILITSRCRVTMYNQKNTLYFNQNNPNNHMYSPNQTFNEIHWTSQELIDTIQNFLQHLGIIEDIYTIYILVS

SEQ ID NO: 5 - DNA, 15328 bp, Artificial Sequence
  Description of Artificial Sequence Synthetic nucleotide sequence of rA2deltaM2-2 cDNA
acgggaaaaaatgcgtacaacaaacttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaacggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgatcacagacatgagaccgttgtcacttgagaccataataacatcactaaccagagacatcataacacacaaatttatatacttgataaatcatgaatgcatagtgagaagacttgatgaaagacaggccacatttacattcctggtcaactatgaaatgaaactattacacaaagtaggaagcactaaatataaaaaatatactgaatacaacacaaaatatggcactttccctatgccaatattcatcaatcatgatgggttcttagaatgcattggcattaagcctacaaagcatactcccataatatacaagtatgatctcaatccataaatttcaacacaatattcacacaatctaaaacaacaactctatgcataactatactccatagtccagatggagcctgaaaattatagtaatttaaaattaaggagagatataagatagaagatggggcaaatacaaagatggctcttagcaaagtcaagttgaatgatacactcaacaaggatcaacttctgtcatccagcaaatacgccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagtcaacacatagcattcatcaatccaacagcccaaaacagtaaccttgcatttaaaaatgaacaacccctacctctttacaacacctcattaacatcccaccatgcaaaccactatccatactataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaagccatgaccaacttaaacagaatcaaagtaaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagtcagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgccggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcattgaaactctcatctataaaccatctcacttacactatttaagtagattcctagtttatagttatataaaacacaattgaatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccgcatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaataagcttcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaacactagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatctttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgtggccggcatggtcccagcctcctcgctggcgccggctgggcaacattccgaggggaccgtcccctcggtaatggcgaatgggacgtcgacagctaacaaagcccgaaggaagtgagttgctgctgccaccgttgagcaataactagcataaccccttggggcctctaaacgggtcttgaggggttttttgctgaaaggagtcgtggagacgttgcgcgcccgcgggtcgacgcggccgcaagcttggcgtaatcatggtcatagctgtttcctgtgtgaaattgttatccgctcacaattccacacaacatacgaccggaagcataaagtgtaaagcctg

SEQ ID NO: 6 - DNA, 17912 bp, Artificial Sequence
  Description of Artificial Sequence Synthetic nucleotide sequence of pUC19+ rA2deltaM2-2 plasmid (used in rescue of recombinant virus)
acgggaaaaaatgcgtacaacaaacttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaacggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgatcacagacatgagaccgttgtcacttgagaccataataacatcactaaccagagacatcataacacacaaatttatatacttgataaatcatgaatgcatagtgagaagacttgatgaaagacaggccacatttacattcctggtcaactatgaaatgaaactattacacaaagtaggaagcactaaatataaaaaatatactgaatacaacacaaaatatggcactttccctatgccaatattcatcaatcatgatgggttcttagaatgcattggcattaagcctacaaagcatactcccataatatacaagtatgatctcaatccataaatttcaacacaatattcacacaatctaaaacaacaactctatgcataactatactccatagtccagatggagcctgaaaattatagtaatttaaaattaaggagagatataagatagaagatggggcaaatacaaagatggctcttagcaaagtcaagttgaatgatacactcaacaaggatcaacttctgtcatccagcaaatacgccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagtcaacacatagcattcatcaatccaacagcccaaaacagtaaccttgcatttaaaaatgaacaacccctacctctttacaacacctcattaacatcccaccatgcaaaccactatccatactataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaagccatgaccaacttaaacagaatcaaagtaaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagtcagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgccggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcattgaaactctcatctataaaccatctcacttacactatttaagtagattcctagtttatagttatataaaacacaattgaatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccgcatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaataagcttcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaacactagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatctttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgtggccggcatggtcccagcctcctcgctggcgccggctgggcaacattccgaggggaccgtcccctcggtaatggcgaatgggacgtcgacagctaacaaagcccgaaggaagtgagttgctgctgccaccgttgagcaataactagcataaccccttggggcctctaaacgggtcttgaggggttttttgctgaaaggagtcgtggagacgttgcgcgcccgcgggtcgacgcggccgcaagcttggcgtaatcatggtcatagctgtttcctgtgtgaaattgttatccgctcacaattccacacaacatacgaccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgttaaaccggccgggtacccccgggtaatacgactcactataggg

(19) United States
(12) Patent Application Publication
(10) Pub. No.: US 2016/0228536 A1
(43) Pub. Date: Aug. 11, 2016

(54) RECOMBINANT RESPIRATORY SYNCYTIAL VIRUS (RSV) AND VACCINES

(71) Applicant: MEDIMMUNE, LLC, Gaithersburg, MD (US)

(72) Inventors: Jeanne Schickli, Sunnyvale, CA (US); Roderick Tang, San Mateo, CA (US); Heather Lawlor, Gaithersburg, MD (US)

(21) Appl. No.: 15/022,825

(22) PCT Filed: Sep. 11, 2014
(86) PCT No.: PCT/US14/55151

§ 371 Date: Mar. 17, 2016

Related U.S. Application Data
(60) Provisional application No. 61/879,270, filed on Sep. 18, 2013.

(51) Int. Cl. A61K 39/155; C07K 14/005; C12N 7/00

(52) U.S. Cl. CPC A61K 39/155; C12N 7/00; C07K 14/005; C12N 2760/18521; C12N 2760/18522; C12N 2760/18562; A61K 2039/5254

(57) ABSTRACT

Described herein is a recombinant respiratory syncytial virus (RSV) having an attenuated phenotype. In one embodiment, recombinant RSV includes an M2-2 protein with a mutation that renders the M2-2 protein inactive or prevents expression of the M2-2 protein. In one embodiment, the amino acid at position 66 in the F subunit has a positively charged side chain. Nucleic acids encoding recombinant RSV are also described, as well as vectors containing the nucleic acids.

### SEQUENCE LISTING

The instant application contains a Sequence Listing which has been submitted in ASCII format via EFS-Web and is hereby incorporated by reference in its entirety. The ASCII copy, created on Aug. 21, 2013, is named DM22-100P1 Sequence Listing.txt and is 51.5 kilobytes in size.

### FIELD OF THE INVENTION

Described herein are mutations that confer attenuated phenotypes important in the production of live attenuated virus vaccines. In one embodiment, recombinant respiratory syncytial viruses that exhibit an attenuated phenotype are provided.

### BACKGROUND OF THE INVENTION

Respiratory syncytial virus (RSV) is the leading cause of serious lower respiratory tract disease in infants and children (Feigen et al., eds., 1987, In: Textbook of Pediatric Infectious Diseases, W B Saunders, Philadelphia at pages 1653-1675; New Vaccine Development, Establishing Priorities, Vol. 1, 1985, National Academy Press, Washington D.C. at pages 397-409; and Ruuskanen et al., 1993, Curr. Probl. Pediatr. 23:50-79). The yearly epidemic nature of RSV infection is evident worldwide, but the incidence and severity of RSV disease in a given season varies by region (Hall, C. B., 1993, Contemp. Pediatr. 10:92-110). In temperate regions of the northern hemisphere, it usually begins in late fall and ends in late spring. Primary RSV infection occurs most often in children from 6 weeks to 2 years of age and uncommonly in the first 4 weeks of life during nosocomial epidemics (Hall et al., 1979, New Engl. J. Med. 300:393-396). Children at increased risk from RSV infection include preterm infants (Hall et al., 1979, New Engl. J. Med. 300:393-396) and children with bronchopulmonary dysplasia (Groothuis et al., 1988, Pediatrics 82:199-203), congenital heart disease (MacDonald et al., 1982, New Engl. J. Med. 307:397-400), congenital or acquired immunodeficiency (Ogra et al., 1988, Pediatr. Infect. Dis. J. 7:246-249; and Pohl et al., 1992, J. Infect. Dis. 165:166-169), and cystic fibrosis (Abman et al., 1988, J. Pediatr. 113:826-830). The fatality rate in infants with heart or lung disease who are hospitalized with RSV infection is 3%-4% (Navas et al., 1992, J. Pediatr. 121:348-354).

RSV infects adults as well as infants and children. In healthy adults, RSV causes predominantly upper respiratory tract disease. It has recently become evident that some adults, especially the elderly, have symptomatic RSV infections more frequently than had been previously reported (Evans, A. S., eds., 1989, Viral Infections of Humans. Epidemiology and Control, 3^{rd} ed., Plenum Medical Book, New York at pages 525-544). Several epidemics also have been reported among nursing home patients and institutionalized young adults (Falsey, A. R., 1991, Infect. Control Hosp. Epidemiol. 12:602-608; and Garvie et al., 1980, Br. Med. J. 281:1253-1254). Finally, RSV may cause serious disease in immunosuppressed persons, particularly bone marrow transplant patients (Hertz et al., 1989, Medicine 68:269-281).

Treatment options for established RSV disease are limited. Severe RSV disease of the lower respiratory tract often requires considerable supportive care, including administration of humidified oxygen and respiratory assistance (Fields et al., eds, 1990, Fields Virology, 2^{nd} ed., Vol. 1, Raven Press, New York at pages 1045-1072). The antiviral agent ribavirin has been approved for treatment of infection (American Academy of Pediatrics Committee on Infectious Diseases, 1993, Pediatrics 92:501-504). It has been shown to be effective in the treatment of RSV pneumonia and bronchiolitis, modifying the course of severe RSV disease in immunocompetent children (Smith et al., 1991, New Engl. J. Med. 325:24-29). However, ribavirin has had limited use because it requires prolonged aerosol administration and because of concerns about its potential risk to pregnant women who may be exposed to the drug during its administration in hospital settings.

A humanized antibody directed to an epitope in the A antigenic site of the F subunit of RSV, SYNAGIS® (palivizumab), is approved for intramuscular administration to pediatric patients for prevention of serious lower respiratory tract disease caused by RSV at recommended monthly doses of 15 mg/kg of body weight throughout the RSV season (November through April in the northern hemisphere). SYNAGIS® is a composite of human (95%) and murine (5%) antibody sequences (Johnson et al., 1997, J. Infect. Diseases 176:1215-1224 and U.S. Pat. No. 5,824,307, the entire contents of which are incorporated herein by reference). The human heavy chain sequence was derived from the constant domains of human IgG₁ and the variable framework regions of the VH genes or Cor (Press et al., 1970, Biochem. J. 117:641-660) and Cess (Takashi et al., 1984, Proc. Natl. Acad. Sci. USA 81:194-198). The human light chain sequence was derived from the constant domain of Cκ and the variable framework regions of the VL gene K104 with Jκ-4 (Bentley et al., 1980, Nature 288:5194-5198). The murine sequences were derived from a murine monoclonal antibody, Mab 1129 (Beeler et al., 1989, J. Virology 63:2941-2950), in a process which involved the grafting of the murine complementarity determining regions into the human antibody frameworks.

A variety of approaches to RSV vaccination have been evaluated over the years including subunits, virus like particles (VLPs) and live-attenuated vaccines (Schickli et al., 2009, Human Vaccines 5, 1-10; Collins & Melero 2011 Virus Res 162, 80-99). The use of non-live RSV vaccine for naive infants is problematic because formalin-inactivated RSV, the first and only non-live RSV vaccine to be tested in naïve infants, was not only ineffective but vaccinated children experienced a more severe disease upon subsequent re-infection with RSV than unvaccinated children, a phenomenon termed RSV enhanced disease (Kapikian et al., 1969 A. J Epidemiol 89:405-421; Kim et al., 1969 Am J Epidemiol 89, 422-434). On the other hand, live-attenuated vaccines are promising, and have been extensively evaluated in RSV-naïve children and infants in the clinic. None of the live-attenuated RSV vaccine candidates tested to date have caused enhanced disease in RSV-naïve infants or children (Karron et al., 2005 J Infec. Dis 191, 1093-1104; Wright et al., 2007 Vaccine 25, 7372-7378). In terms of immunogenicity, live-attenuated virus is expected to most closely mimic the natural route of infection and, in turn, stimulate protective mucosal, humoral and cellular immune responses.

### SUMMARY OF THE INVENTION

Described herein is a recombinant respiratory syncytial virus (RSV) having an attenuated phenotype. In one embodiment, recombinant RSV also includes an M2-2 protein having a mutation that renders the M2-2 protein inactive or prevents expression of the M2-2 protein. In one embodiment, recombinant RSV also includes a F subunit in which a naturally occurring amino acid found at position 66 is artificially substituted with an amino acid residue having a positive side chain, for example, arginine (R), lysine (K) or histidine (H). In one embodiment, the amino acid at position 66 in the F subunit has a negatively charged side chain, such as Glutamic acid (E).

In one embodiment, the M2-2 protein has an amino acid sequence that is at least about 95% identical to the amino acid sequence of the M2-2 protein shown in SEQ ID NO: 4. In one embodiment, the M2-2 protein has an amino acid sequence that includes a deletion of at least about 5 amino acid residues from the amino acid sequence of the M2-2 protein shown in SEQ ID NO:4. In another embodiment, the M2-2 protein has an amino acid sequence that includes a deletion of at least about 5% of the amino acids from the amino acid sequence of the M2-2 protein shown in SEQ ID NO: 4. In one embodiment, one or more amino acids are deleted from the N-terminus. In another embodiment, one or more amino acids are deleted from the C-terminus. Nucleic acids encoding recombinant respiratory syncytial virus (RSV) having an attenuated phenotype are also provided. The nucleic acid can be DNA or RNA, for example, mRNA. In one embodiment, the nucleic acid is included within a vector.

Also described herein is a respiratory syncytial virus (RSV) vaccine that includes an immunologically effective amount of recombinant RSV, as well as pharmaceutical compositions that include recombinant RSV and methods of stimulating a protective immune response, preventing disease caused by RSV, inducing neutralizing antibodies against RSV and/or reducing RSV viral titers, wherein the methods include administering an immunologically effective amount of recombinant RSV to a mammal, for example, a human. In one embodiment, recombinant RSV is administered in a single dose. In another embodiment, recombinant RSV is administered in more than one dose.

### BRIEF DESCRIPTION OF THE DRAWINGS

FIG. 1. Multi-cycle growth curves of rA2ΔM2-2 viruses in 3 cell lines. HEp-2 (a), Vero (b) or SF Vero cells (c) cells were infected at an MOI=0.1. Samples were collected at 24 hr intervals and titered by plaque assay.

FIG. 2. Syncytium formation of rA2ΔM2-2 viruses in Vero cells. Cells were infected at MOI=0.1 with rA2ΔM2-2(MEDI) (a) or rA2ΔM2-2(NIH) (b). Images were captured at 48 hpi at 40× magnification on a Nikon Eclipse TS 100 microscope.

FIG. 3. Deletion of M2-2 ORF from RSVA2. The M2 gene has two overlapping reading frames, M2-1 and M2-2. The translational stop of the M2-1 ORF is underlined and marked by an asterisk below the TGA codon. The translational stop of the M2-2 ORF is indicated by an asterisk above the TAA codon.

FIG. 4. Multi-cycle growth curve of rA2ΔM2-2(MEDI) variants. SF Vero cells in 6-well plates were infected with each virus at MOI=0.1 PFU/cell. Samples were harvested at 24 hr intervals and titered by plaque assay.

FIG. 5. Effect of amino acid substitutions in RSV F at position 66. (a) Linear representation of RSV F protein. F2 fragment extends from aa22 to aa109. F1 fragment extends from aa136-574. Potential N-glycosylation sites are denoted by closed triangles. SP=signal peptide; HRA=heptad repeat A; FP=fusion peptide; HRB=heptad repeat B; TM=transmembrane region; CT=cytoplasmic tail. SP is cleaved at aa22. Furin cleavage sites are at aa109 and aa136. Amino acid sequences flanking K66E and Q101P are shown below the diagram and sites of aa differences are underlined. (b) Vero cells were transfected with either pF/66K or pF/66E. Cells were fixed at 24 h intervals and immunostained with antibody directed to RSV F to observe relative formation of syncytia. (c) Vero cells were transfected with the variant pCMV/RSVF plasmids. The amino acids at position 66 are denoted within each panel. Cells were fixed at 48 h and immunostained with antibody directed to RSV F to observe relative formation of syncytia. (d) Western blot of lysates from Vero cells transfected with the variant pCMV/RSVF plasmids. Letters above lanes denote amino acid at position 66. Western blots were probed with antibody directed to either RSV F and normalized by blotting with β-actin. (e) Expression of RSV F on surface of transfected 293T cells as determined by FACS. Letters on x-axis denote amino acid at position 66.

FIG. 6. Structure of the RSV F homotrimer. F2 fragment within each RSV F monomer is a different shade of red. F1 fragment within each RSV F monomer is a different shade of blue. Amino acid 66 is marked in yellow. (a) Pre-fusion model based on PDB 4JHW (McLellan et al., 2013). (b) Post-fusion model based on PDB 3RRT (McLellan et al., 2011).

Table 1 discloses the genetic differences between rA2ΔM2-2(MEDI) and rA2ΔM2-2(NIH). Numbering is based on rA2ΔM2-2(MEDI) sequence. nt=nucleotide, aa=amino acid.

### DETAILED DESCRIPTION

### 1. Definitions

The term “about” refers to variation in the numerical quantity that can occur, for example, through typical measuring and handling procedures used for making compounds, compositions, concentrates or formulations; through inadvertent error in these procedures; through differences in the manufacture, source, or purity of starting materials or ingredients used to carry out the methods, and similar considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

As used in this specification and the appended claims, the singular forms “a,” “an” and “the” include plural referents unless the context clearly dictates otherwise. Thus, for example, reference to “a virus” includes a plurality of viruses; reference to “a host cell” includes mixtures of host cells, and the like.

An “amino acid sequence” is a polymer of amino acid residues (a protein, polypeptide, etc.) or a character string representing an amino acid polymer, depending on context.

As used herein, an “antibody” is a protein that includes one or more polypeptides substantially or partially encoded by immunoglobulin genes or fragments of immunoglobulin genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is a tetramer. Each tetramer includes two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains, respectively. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1999), for a more detailed description of other antibody fragments). Antibodies include, e.g., polyclonal antibodies, monoclonal antibodies, multiple or single chain antibodies, including single chain Fv (sFv or scFv) antibodies in which a variable heavy and a variable light chain are joined together (directly or through a peptide linker) to form a continuous polypeptide, and humanized or chimeric antibodies.

An “artificial mutation” is a mutation introduced by human intervention, e.g., under laboratory conditions. Thus, an “artificially mutated” nucleotide is a nucleotide that has been mutated as a result of human intervention and an “artificially altered” amino acid residue is a residue that has been altered as a result of human intervention. For example, a wild type protein can be “artificially altered” by artificially mutating the gene encoding that protein.

An RSV “having an attenuated phenotype” or an “attenuated” RSV exhibits a substantially lower degree of virulence as compared to a non-attenuated or wild-type virus. An attenuated RSV typically exhibits a slower growth rate and/or a reduced level of replication, for example, a peak titer that is at least about ten fold, or at least about one hundred fold less than that of a non-attenuated or wild-type RSV.

As used herein, the term “effective amount” refers to an amount of antigen necessary or sufficient to realize a desired clinical outcome. The term “effective dose” generally refers to the amount of an antigen that can induce a protective immune response sufficient to induce immunity to prevent and/or ameliorate an infection or disease, and/or to reduce at least one symptom of an infection or disease. The term a “therapeutically effective amount” refers to an amount which provides a therapeutic effect for a given condition and administration regimen.

“Expression of a gene” or “expression of a nucleic acid” refers to transcription of DNA into RNA, translation of RNA into a polypeptide, or both transcription and translation, as indicated by the context.

The term “gene” is used broadly to refer to a nucleic acid associated with a biological function. Thus, genes include coding sequences and/or the regulatory sequences required for their expression. The term “gene” applies to a specific genomic sequence, as well as to a cDNA or an mRNA encoded by that genomic sequence. Genes also include non-expressed nucleic acid segments that, for example, form recognition sequences for other proteins. Non-expressed regulatory sequences include “promoters” and “enhancers,” to which regulatory proteins such as transcription factors bind, resulting in transcription of adjacent or nearby sequences.

The term “host cell” refers to a cell which contains a heterologous nucleic acid, such as a vector, and supports the replication and/or expression of the nucleic acid. Host cells can be prokaryotic cells such as E. coli, or eukaryotic cells such as yeast, insect, amphibian, avian or mammalian cells, including human cells, for example, HEp-2 cells and Vero cells.

As used herein, the terms “immunogen” or “antigen” refer to substances such as proteins, peptides, and nucleic acids that are capable of eliciting an immune response. Both terms also encompass epitopes, and are used interchangeably. As use herein, the term “immunogenic formulation” refers to a preparation which, when administered to a vertebrate, e.g. a mammal, will induce an immune response.

An “immunologically effective amount” of RSV is an amount sufficient to enhance a mammal's immune response against a subsequent exposure to RSV. Levels of induced immunity can be monitored, for example, by measuring amounts of neutralizing secretory and/or serum antibodies by methods such as plaque neutralization, complement fixation, enzyme-linked immunosorbent, or microneutralization assays.

The term “introduced” when referring to a heterologous or isolated nucleic acid refers to the transfer of a nucleic acid into a eukaryotic or prokaryotic cell where the nucleic acid can be incorporated into the genome of the cell, converted into an autonomous replicon, or transiently expressed. The term includes such methods as “infection,” “transfection,” “transformation” and “transduction.” A variety of methods can be employed to introduce nucleic acids into host cells, including electroporation, calcium phosphate precipitation, lipid mediated transfection, lipofection, etc.

The term “isolated” refers to a biological material, such as a virus, a nucleic acid or a protein, which is substantially free from components that normally accompany or interact with it in its naturally occurring environment. The isolated material may include material not found with the material in its natural environment. For example, if the material is in its natural environment, such as a cell, the material may have been placed at a location in the cell not native to a material found in that environment. For example, a naturally occurring nucleic acid can be considered isolated if it is introduced by non-naturally occurring means to a locus of the genome not native to that nucleic acid. Such nucleic acids are also referred to as “heterologous” nucleic acids. An isolated virus, for example, may be in an environment (e.g., a cell culture system, or purified from cell culture) other than the native environment of a wild-type virus (e.g., the nasopharynx of an infected mammal).

The term “nucleic acid” or “polynucleotide” encompasses any physical string of monomer units that can be corresponded to a string of nucleotides, including a polymer of nucleotides (e.g., a typical DNA or RNA polymer), PNAs, modified oligonucleotides (e.g., oligonucleotides having bases that are not typical to biological RNA or DNA in solution, such as 2′-O-methylated oligonucleotides), and the like. A nucleic acid can be single-stranded or double-stranded. Unless otherwise indicated, a nucleic acid sequence encompasses complementary sequences, in addition to the sequence explicitly indicated.

An “open reading frame” or “ORF” is a possible translational reading frame of DNA or RNA, which is capable of being translated into a polypeptide. That is, the reading frame is not interrupted by stop codons. However, it should be noted that the term ORF does not necessarily indicate that the polynucleotide is, in fact, translated into a polypeptide.

The phrase “percent identical” or “percent identity” refers to the similarity between at least two different sequences. Percent identity can be determined by standard alignment algorithms, for example, the Basic Local Alignment Search Tool (BLAST) described by Altshul et al. ((1990) J. Mol. Biol., 215: 403-410); the algorithm of Needleman et al. ((1970) J. Mol. Biol., 48: 444-453); or the algorithm of Meyers et al. ((1988) Comput. Appl. Biosci., 4: 11-17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. Percent identity is usually calculated by comparing sequences of similar length.

As used herein, “pharmaceutical composition” refers to a composition that includes a therapeutically effective amount of attenuated RSV together with a pharmaceutically acceptable carrier and, if desired, one or more diluents or excipients. As used herein, the term “pharmaceutically acceptable” means that it is approved by a regulatory agency of a Federal or a state government or listed in the U.S. Pharmacopia, European Pharmacopia or other generally recognized pharmacopia for use in mammals, and more particularly in humans.

As used herein, the term “pharmaceutically acceptable vaccine” refers to a formulation that contains an immunogen in a form that is capable of being administered to a vertebrate and that induces a protective immune response sufficient to induce immunity to prevent and/or ameliorate an infection or disease, and/or to reduce at least one symptom of an infection or disease. In one embodiment, the vaccine includes attenuated RSV as an immunogen and prevents or reduces at least one symptom of RSV infection in a subject. Symptoms of RSV are well known in the art and include, but are not limited to, rhinorrhea, sore throat, headache, hoarseness, cough, sputum, fever, rales, wheezing, and dyspnea. Thus, in one embodiment, the method can include prevention or reduction of at least one symptom associated with RSV infection. A reduction in a symptom may be determined subjectively or objectively, e.g., self assessment by a subject, by a clinician's assessment or by conducting an appropriate assay or measurement (e.g. body temperature), including, e.g., a quality of life assessment, a slowed progression of a RSV infection or additional symptoms, a reduced, severity of a RSV symptoms or a suitable assays (e.g. antibody titer and/or T-cell activation assay).

A “polypeptide” is a polymer having two or more amino acid residues (e.g., a peptide or a protein). The polymer may also include modifications such as glycosylation. The amino acid residues of the polypeptide can be natural or non-natural and can be unsubstituted, unmodified, substituted or modified.

As used herein, the phrase “protective immune response” or “protective response” refers to an immune response mediated by antibodies against an infectious agent or disease, which is exhibited by a vertebrate, for example, a human, that prevents or ameliorates an infection or reduces at least one disease symptom thereof. In one embodiment, administration of an attenuated RSV vaccine described herein elicits a protective immune response in a patient. In one embodiment, the attenuated RSV vaccines described herein can stimulate the production of antibodies that, for example, neutralize infectious agents, block infectious agents from entering cells, block replication of the infectious agents, and/or protect host cells from infection and destruction. The term can also refer to an immune response that is mediated by T-lymphocytes and/or other white blood cells against an infectious agent or disease, exhibited by a vertebrate, for example, a human, that prevents or ameliorates infection or disease, or reduces at least one symptom thereof.

The term “recombinant” indicates that the material has been artificially or synthetically altered by human intervention. The alteration can be performed on the material within, or removed from, its natural environment or state. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus” is produced by the expression of a recombinant nucleic acid.

As used herein, the term “vaccine” refers to a preparation of dead or weakened pathogens, or antigenic determinants derived from a pathogen, wherein the preparation is used to induce formation of antibodies or immunity against the pathogen. In addition, the term “vaccine” can also refer to a suspension or solution of an immunogen that is administered to a vertebrate, for example, to produce protective immunity.

The term “variant” with respect to a polypeptide refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Variants can also include amino acid deletion or insertion, or both. Guidance in determining which amino acid residues can be substituted, inserted, or deleted without eliminating biological or immunological activity can be found using computer programs well known in the art, for example, DNASTAR software.

The term “vector” refers to the means by which a nucleic acid can be propagated and/or transferred between organisms, cells, or cellular components. Vectors include, but are not limited to, plasmids, viruses, bacteriophage, pro-viruses, phagemids, transposons, and artificial chromosomes, that replicate autonomously or can integrate into a chromosome of a host cell. A vector can also be a naked RNA polynucleotide, a naked DNA polynucleotide, a polynucleotide that includes both DNA and RNA within the same strand, a poly-lysine-conjugated DNA or RNA, a peptide-conjugated DNA or RNA, a liposome-conjugated DNA, or the like, that are not autonomously replicating. An “expression vector” is a vector, such as a plasmid, which is capable of promoting expression as well as replication of a nucleic acid incorporated therein. Typically, the nucleic acid to be expressed is “operably linked” to a promoter and/or enhancer, and is subject to transcription regulatory control by the promoter and/or enhancer.

As used herein, the term “vertebrate” or “subject” or “patient” refers to any member of the subphylum cordata, including, without limitation, humans and other primates, including non-human primates such as chimpanzees and other apes and monkey species. Farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like are also non-limiting examples. The terms “mammals” and “animals” are included in this definition. Both adult and newborn mammals are intended to be covered. In particular, infants and young children are appropriate subjects or patients for a RSV vaccine.

As used herein, “viral fusion protein” or “fusion protein” or “F subunit” refers to any viral fusion protein, including but not limited to, a native viral fusion protein or a soluble viral fusion protein, including recombinant viral fusion proteins, synthetically produced viral fusion proteins, and viral fusion proteins extracted from cells. As used herein, “native viral fusion protein” refers to a viral fusion protein encoded by a naturally occurring viral gene or viral RNA. Viral fusion proteins include related proteins from different viruses and viral strains including, but not limited to viral strains of human and non-human categorization. Viral fusion proteins include type I and type II viral fusion proteins. Numerous RSV-Fusion proteins have been described and are known to those of skill in the art. As used herein, the term “recombinant viral fusion protein” refers to a viral fusion protein derived from an engineered nucleotide sequence and produced in an in vitro and/or in vivo expression system.

### 2. Respiratory Syncytial Virus (RSV)

Human respiratory syncytial virus (RSV) is a member of the family Paramyxoviridae, subfamily Pneumovirinae and genus Pneumovirus. RSV is an enveloped virus having a single-stranded nonsegmented negative-sense RNA genome of 15,221 nucleotides (Collins, 1991, In The paramyxoviruses pp. 103-162, D. Kingsbury (ed.) Plenum Press, New York), which encodes three transmembrane structural proteins (F, G and SH), two matrix proteins (M and M2), three nucleocaspid proteins (N, P and L) and two nonstructural proteins (NS1 and NS2). The genome contains a 44 nucleotide leader sequence at the 3′ termini followed by the encoded proteins (NS1-NS2-N-P-M-SH-G-F-M2-L) and a 155 nucleotide trailer sequence at the 5′ termini (Collins. 1991, In The paramyxoviruses pp. 103-162, D. Kingsbury (ed.) Plenum Press, New York). RSV is divided into two subgroups, A and B, which are differentiated primarily on the variability of the G gene and encoded protein. Many RSV strains are known, and include, for example, Human strains such as A2, Long, ATCC VR-26, 19, 6265, E49, E65, B65, RSB89-6256, RSB89-5857, RSB89-6190, and RSB89-6614; or Bovine strains such as ATue51908, 375, and A2Gelfi; or Ovine strains.

Fusion of infected cells is a hallmark of all Paramyxoviruses (Dutch et al. 2000 Biosci. Rep. 20:597-612). The fused mass of cells is called “syncytium,” from which RSV derives its middle name. Although multiple viral proteins may be required for syncytium induction, the fusion protein F is the central mediator of the process. It is believed that F subunit expression on the surface of the virus causes the cell membranes on nearby cells to merge, forming syncytia.

The F subunit is a type I transmembrane surface protein that has an N-terminal cleaved signal peptide and a membrane anchor near the C-terminus. In nature, the RSV-F subunit is expressed as a single inactive 574 amino acid precursor designated F₀. In vivo, F₀ oligomerizes in the endoplasmic reticulum and is proteolytically processed by an endoprotease to yield a linked heterodimer containing two disulfide-linked subunits, F₁ and F₂. The smaller of these fragments is termed F₂ and originates from the N-terminal portion of the F₀ precursor. The N-terminus of the F₁ subunit that is created by cleavage contains a hydrophobic domain (the fusion peptide), which associates with the host cell membrane and promotes fusion of the membrane of the virus, or an infected cell, with the target cell membrane. Frequently, the F-protein is a trimer or multimer of F₁/F₂ heterodimers. The nucleic acid and amino acid sequences for the F₀ protein from the A2 strain are shown in SEQ ID NOs: 1 and 2, respectively.

The M2-2 gene is thought to govern the transition from transcription to production of genomic RNA. The M2 gene is located between the genes encoding the F and L proteins and encodes two putative proteins: M2-1 and M2-2. The 22-kDa M2-1 protein is encoded by the 5′-proximal open reading frame of the M2 mRNA, and its open reading frame partially overlaps the second, M2-2, open reading frame by 31 nucleotides (Collins et al. 1985. J. Virol. 54:65-71). The M2-1 protein has been shown to be a transcriptional processivity factor that is involved in RNA transcription elongation (Collins et al. 1996. PNAS USA 93:81-85). The M2-1 protein also decreases RNA transcription termination and facilitates read-through of RNA transcription at each gene junction (Hardy et al. 1999. J. Virol. 73:170-176; Hardy and Wertz. 1998. J. Virol. 72:520-526). The M2-2 polypeptide contains 90 amino acids and down-regulates RSV RNA transcription and replication in a minigenome model system (Collins et al. 1996. PNAS USA, 93:81-85). The nucleic acid and amino acid sequences for M2-2 from A2 strain of RSV are shown in SEQ ID NOs: 3 and 4, respectively.

### 3. Attenuated Virus

As used herein, the term “attenuated” refers to a strain of a virus whose pathogenicity and/or virulence has been reduced as compared to a non-attenuated or wild-type virus, such that it can be used to stimulate an immune response without causing symptoms of viral infection or disease, or at least in which such symptoms are reduced. An attenuated virus can be used to make a vaccine that is capable of stimulating an immune response in an immunized animal without causing illness. For example, attenuated virus may exhibit a substantially lower degree of virulence as compared to a wild-type virus. For example, attenuated RSV may exhibit one or more of the following: a slower growth rate, reduction in syncytium formation, or reduced fusogenicity such that one or more symptoms of viral infection are reduced or do not occur in an immunized mammal.

Attenuated virus can include live virus that has been subjected to one or more mutations that render it less virulent. Mutations include, for example, single nucleotide changes, site-specific mutations, insertions, substitutions, deletions, or rearrangements of the viral genome. Mutations may affect a single amino acid, a small segment of the genome, for example, at least about 1, 5, 10, 15, 20 or 25 nucleotides and up to about 30, 35, 40, 45 or 50 nucleotides, or a larger segment of the genome, for example, at least about 50, 55, 60, 65, 70 or 75 nucleotides and up to about 75, 80, 85, 90, 95, 100 or more nucleotides, depending on the nature of the mutation. Mutations can also be introduced upstream or downstream of an existing cis-acting regulatory element in order to ablate its activity, thus resulting in an attenuated phenotype. Alternately, a non-coding regulatory region of a virus can be altered to down-regulate any viral gene, e.g. reduce transcription of its mRNA and/or reduce replication of vRNA (viral RNA), so that an attenuated virus is produced.

In one embodiment, live attenuated RSV vaccines are provided. In one embodiment, genetically engineered recombinant respiratory syncytial virus (RSV) and viral vectors that express one or more mutated viral genes are provided. In one embodiment, recombinant negative strand viral RNA templates are provided, wherein the templates may be used with viral RNA-directed RNA polymerase to express gene products in appropriate host cells. The RNA templates may be prepared by transcription of appropriate DNA sequences using a DNA-directed RNA polymerase such as bacteriophage T7, T3 or Sp6 polymerase. The recombinant RNA templates may be used to transfect continuous/transfected cell lines that express the RNA-directed RNA polymerase proteins. Recombinant RSV can include any species subgroup and/or strain of RSV. In one embodiment, recombinant RSV includes a human RSV of subgroup A, subgroup B or a chimera thereof.

Typically, recombinant RSV used in a vaccine is sufficiently attenuated such that symptoms of infection, or at least symptoms of serious infection, will not occur in most mammals immunized or otherwise infected with the attenuated RSV. In some instances, the attenuated RSV can still be capable of producing symptoms of mild illness, for example, mild upper respiratory illness and/or of dissemination to unvaccinated mammals. However, virulence is sufficiently abrogated such that severe lower respiratory tract infections do not typically occur in the vaccinated or incidental host.

### 4. M2-2 Deletion

One of the major challenges in developing a safe and effective live attenuated RSV vaccine is maintaining the delicate balance between limiting virus replication in the host and delivering an antigen load sufficient to induce a protective immune response. Many live attenuated RSV vaccine candidates rely on point mutations to attenuate growth. Reversion of these point mutations can result in partial reversion of the attenuation phenotype as was observed in the rA2cp248/404/1030ΔSH clinical trial (Karron et al., 2005, J. Infect. Dis. 191:1093-1104). Partial reversion of the attenuation phenotype raises concerns about transmission of less attenuated virus to vulnerable contacts of vaccines.

In one embodiment, a recombinant respiratory syncytial virus (RSV) polypeptide that exhibits an attenuated phenotype is provided, wherein recombinant RSV includes one or more artificially altered amino acids, for example, at least one deleted, inserted and/or substituted amino acid. In one embodiment, recombinant RSV includes one or more mutations that inactivate the M2-2 gene product and/or ablates expression of the M2-2 gene. It is believed that inactivation and/or deletion of M2-2 results in an imbalance which favors transcription over replication, resulting in increased viral protein expression. Advantageously, RSV M2-2 mutants demonstrate attenuated growth, but do not substantially compromise the expression level of viral antigens, thereby helping to maintain a high level of antigen load. In one embodiment, recombinant RSV has a M2-2 amino acid sequence that is at least about 75%, 80%, 85%, 90%, 95%, 96%, 97%, 98%, or 99% identical to the amino acid sequence of the M2-2 protein shown in SEQ ID NO: 4.

In another embodiment, nucleic acids that encode recombinant RSV that exhibits an attenuated phenotype are provided. In one embodiment, the nucleic acid encodes recombinant RSV that includes one or more artificially altered amino acids, for example, at least one deleted, inserted and/or substituted amino acid. In one embodiment, the nucleic acid encodes one or more mutations that inactivate the M2-2 gene product and/or ablate expression of the M2-2 gene. In one embodiment, the nucleic acid encoding recombinant RSV has a sequence that is at least about 75%, 80%, 85%, 90%, 95%, 96%, 97%, 98%, or 99% identical to the nucleic acid sequence shown in SEQ ID NO: 3. In one embodiment, the nucleic acid is DNA, for example, cDNA. In another embodiment, the nucleic acid is RNA, for example, mRNA. In one embodiment, the nucleic acid is included within a vector, for example, a plasmid.

In one embodiment, recombinant RSV includes a mutation in which at least a part of the M2-2 protein is deleted. Advantageously, M2-2 deletion mutants result in a virus having an attenuated phenotype which is less likely to revert than point mutations. In one embodiment, recombinant RSV includes a deletion of at least about 5, 10, 15, 20, 25, 30, 35, 40, or 45 amino acid residues from an amino acid sequence of an M2-2 protein shown in SEQ ID NO: 4, and up to at least about 50, 55, 60, 65, 70, 75, 80, 85, or 90 amino acid residues from an amino acid sequence of an M2-2 protein shown in SEQ ID NO:4, wherein the deletion is sufficient to render the M2-2 protein inactive and/or prevent expression of the M2-2 protein. In one embodiment, recombinant RSV has an amino acid sequence that is at least about 75%, 80%, 85%, 90%, 95%, 96%, 97%, 98%, or 99% identical to an amino acid sequence of an M2-2 protein shown in SEQ ID NO: 4 and includes a deletion of at least about 5, 10, 15, 20, 25, 30, 35, 40, or 45 amino acid residues from an amino acid sequence of the M2-2 protein shown in SEQ ID NO: 4, and up to at least about 50, 55, 60, 65, 70, 75, 80, 85, or 90 amino acid residues from an amino acid sequence of the M2-2 protein shown in SEQ ID NO:4, wherein the deletion is sufficient to render the M2-2 protein inactive and/or prevent expression of the M2-2 protein. In one embodiment, one or more amino acids are deleted from the N-terminus of the M2-2 amino acid sequence. In another embodiment, one or more amino acids are deleted from the C-terminus of the M2-2 amino acid sequence.

In one embodiment, recombinant RSV includes a deletion of at least about 5%, 10%, 15%, 20%, 25%, 30%, 35%, 40%, 45% or 50% of the amino acid residues of an amino acid sequence of a M2-2 protein shown in SEQ ID NO:4 and up to at least about 55%, 60%, 65%, 70%, 75%, 80%, 85%, 90%, 95% or 100% of the amino acid residues of the M2-2 protein shown in SEQ ID NO:4, wherein the deletion is sufficient to render the M2-2 protein inactive and/or prevent expression of the M2-2 protein. In one embodiment, recombinant RSV has an amino acid sequence that is at least about 75%, 80%, 85%, 90%, 95%, 96%, 97%, 98%, or 99% identical to an amino acid sequence of an M2-2 protein shown in SEQ ID NO: 4 and includes a deletion of at least about 5%, 10%, 15%, 20%, 25%, 30%, 35%, 40%, 45% or 50% of the amino acid residues of an amino acid sequence of a M2-2 protein shown in SEQ ID NO:4 and up to at least about 55%, 60%, 65%, 70%, 75%, 80%, 85%, 90%, 95% or 100% of the amino acid residues of the M2-2 protein shown in SEQ ID NO:4, wherein the deletion is sufficient to render the M2-2 protein inactive and/or prevent expression of the M2-2 protein. In one embodiment, one or more amino acids are deleted from the N-terminus of the M2-2 amino acid sequence. In another embodiment, one or more amino acids are deleted from the C-terminus of the M2-2 amino acid sequence.

In one embodiment, the deletion in the M2-2 protein is sufficient to up regulate viral transcription. In one embodiment, the deletion in the M2-2 protein is sufficient to alter the ratio between replication and transcription. As used herein, the term “replication” refers to the formation of copies of the viral genome. The genome copies are then packaged into viral particles which exit the host cell and continue the infection process. As used herein, the term “transcription” refers to transcription from the negative-stranded genome by the viral RNA-dependent RNA polymerase to yield mRNAs that encode the various viral proteins.

In one embodiment, a polynucleotide encoding recombinant RSV that includes a mutation in which at least a part of the M2-2 protein is deleted is provided. In one embodiment, the polynucleotide encodes recombinant RSV in which at least about 5, 10, 15, 20, 25, 30, 35, 40, or 45 amino acid residues from an amino acid sequence of an M2-2 protein shown in SEQ ID NO:4 and up to at least about 50, 55, 60, 65, 70, 75, 80, 85, or 90 amino acid residues from an amino acid sequence of an M2-2 protein shown in SEQ ID NO:4 are deleted, wherein the deletion is sufficient to render the M2-2 protein inactive and/or prevent expression of the M2-2 protein. In one embodiment, the polynucleotide encoding recombinant RSV has an nucleic acid sequence that is at least about 75%, 80%, 85%, 90%, 95%, 96%, 97%, 98%, or 99% identical to a nucleic acid sequence of SEQ ID NO: 3 and encodes recombinant RSV in which at least about 5, 10, 15, 20, 25, 30, 35, 40, or 45 amino acid residues from an amino acid sequence of an M2-2 protein shown in SEQ ID NO:4 and up to at least about 50, 55, 60, 65, 70, 75, 80, 85, or 90 amino acid residues from an amino acid sequence of an M2-2 protein shown in SEQ ID NO:4 are deleted, wherein the deletion is sufficient to render the M2-2 protein inactive and/or prevent expression of the M2-2 protein. In one embodiment, one or more amino acids are deleted from the N-terminus of the M2-2 amino acid sequence. In another embodiment, one or more amino acids are deleted from the C-terminus of the M2-2 amino acid sequence.

In one embodiment, the polynucleotide encodes recombinant RSV that includes a deletion of at least about 5%, 10%, 15%, 20%, 25%, 30%, 35%, 40%, 45% or 50% of the amino acid residues of an amino acid sequence of an M2-2 protein shown in SEQ ID NO: 4 and up to at least about 55%, 60%, 65%, 70%, 75%, 80%, 85%, 90%, 95% or 100% of the amino acid residues of an amino acid sequence of an M2-2 protein shown in SEQ ID NO:4, wherein the deletion is sufficient to render the M2-2 protein inactive and/or prevent expression of the M2-2 protein. In one embodiment, the polynucleotide encoding recombinant RSV has an nucleic acid sequence that is at least about 75%, 80%, 85%, 90%, 95%, 96%, 97%, 98%, or 99% identical to a nucleic acid sequence of SEQ ID NO: 3 and encodes recombinant RSV that includes a deletion of at least about 5%, 10%, 15%, 20%, 25%, 30%, 35%, 40%, 45% or 50% of the amino acid residues of an amino acid sequence of an M2-2 protein shown in SEQ ID NO: 4 and up to at least about 55%, 60%, 65%, 70%, 75%, 80%, 85%, 90%, 95% or 100% of the amino acid residues of an amino acid sequence of an M2-2 protein shown in SEQ ID NO:4, wherein the deletion is sufficient to render the M2-2 protein inactive and/or prevent expression of the M2-2 protein. In one embodiment, one or more amino acids are deleted from the N-terminus of the M2-2 amino acid sequence. In another embodiment, one or more amino acids are deleted from the C-terminus of the M2-2 amino acid sequence.

In one embodiment, the deletion encoded by the polynucleotide is sufficient to up regulate viral transcription. In one embodiment, the deletion encoded by the M2-2 protein is sufficient to alter the ratio between replication and transcription.

### 5. K66 Mutation

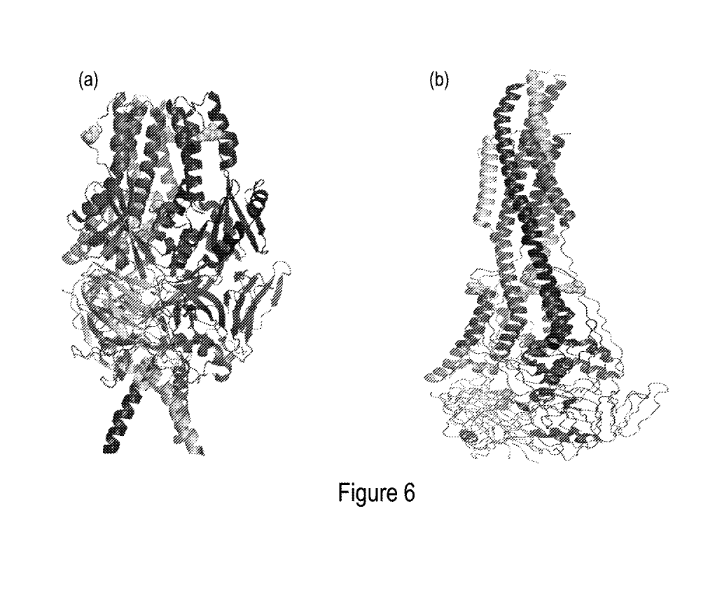

In one embodiment, recombinant RSV that exhibits an attenuated phenotype is provided, wherein the virus includes an F subunit having at least one artificially mutated amino acid, for example, at least one deleted, inserted and/or substituted amino acid. In one embodiment, recombinant RSV includes an F subunit having at least one substituted amino acid. In a more particular embodiment, recombinant RSV includes an F subunit in which the naturally occurring amino acid found at position 66 in a wild-type sequence has been mutated. In one embodiment, recombinant RSV includes an F subunit in which the naturally occurring amino acid found at position 66 in a wild-type sequence has been artificially mutated. Amino acid positions referred to herein are given in reference to the F subunit precursor polypeptide (F₀) sequence shown in SEQ ID NO:2. However, it should be noted that, since F₂ corresponds to approximately the first 109 amino acids of the F₀ precursor, the amino acid found at position 66 of F₀ also refers to the amino acid at position 66 in F₂, and can be used interchangeably. For the sake of convenience and consistency, the amino acid at this position will be referred to as the amino acid found at position 66 of the F subunit. Amino acid 66 is located in the F₂ fragment of the fully processed RSV F and has been mapped to a position on the outer surface of the homotrimer near the mid-span of the fully extended HRA (Swanson et al. 2011. PNAS USA, 108:9619-9624) (FIG. 6).

In one embodiment, recombinant RSV includes an F subunit that has an amino acid sequence that is at least about 75%, 80%, 85%, 90%, 95%, 96%, 97%, 98%, or 99% identical to an amino acid sequence of an F subunit shown in SEQ ID NO: 2. In one embodiment, recombinant RSV is encoded by a nucleic acid sequence that is at least about 75%, 80%, 85%, 90%, 95%, 96%, 97%, 98%, or 99% identical to a nucleic acid sequence encoding F₀ shown in SEQ ID NO: 1.

In one embodiment, recombinant RSV includes an F subunit having at least one substituted amino acid residue at position 66. The phrase “a substituted amino acid” refers to a sequence in which an amino acid residue occupying a particular position in a protein is replaced by another amino acid. For example, in F subunit shown in SEQ ID NO:2, the amino acid residue at position 66 is lysine (K), which can be denoted Lysine66. An amino acid substitution can be abbreviated using standard notation in which the ancestral amino acid is reported in front of the residue location and the mutant (or substituted) amino acid follows the residue location. For example, a mutant in which the lysine (K) at position 66 in the protein is substituted with Glutamic Acid (E) can be denoted by the abbreviation Lysine66Glutamic Acid or K66E.

In one embodiment, the F subunit includes an artificially substituted amino acid having a positive side chain at residue 66. Amino acids can be sorted into 4 groups based on the nature of their side chain: (1) hydrophobic, (2) polar but uncharged, (3) basic, and (4) acidic. Of the 20 common amino acids, amino acids with hydrophobic side chains include glycine (Gly), alanine (Ala), valine (Val), leucine (Leu), isoleucine (Ile), proline (Pro), phenylalanine (Phe), methionine (Met), and tryptophan (Trp). Amino acids with side chains that are polar but not charged include serine (Ser), threonine (Thr), cysteine (Cys), asparagine (Asn), glutamine (Gln), and tyrosine (Tyr). Amino acids that have side chains that are fully protonated (i.e., have a positive charge) at neutral pH include arginine (Arg), lysine (Lys), and histidine (His). Positive amino acids are said to have “basic” side chains. Amino acids with side chains that are ionized (and therefore have negative charge) at neutral pH include aspartic acid or aspartate (Asp) and glutamic acid or glutamate (Glu). Negative amino acids are said to have “acidic” side chains. The term “neutral pH” refers to a pH that is around 7, for example, between 6 and 8 or between 6.5 and 7.5 or between 7.0 and 7.5 or between 7.3 and 7.4.

The inventors have found that inclusion of a positively charged residue at position 66 of the F₀ sequence shown in SEQ ID NO:2 results in an attenuated virus with improved growth when compared to a virus having a non-positive amino acid at position 66, such as Glutamic Acid (E). In one embodiment, recombinant RSV F subunit includes a positively charged amino acid such as Lysine (K) at position 66 of the F₀ sequence shown in SEQ ID NO:2. In another embodiment, the positively charged amino acid residue at position 66 of the F₀ sequence shown in SEQ ID NO:2 is not Lysine (K). In one embodiment, the Lysine found at position 66 in the F₀ sequence shown in SEQ ID NO:2 is substituted with an amino acid having a negatively charged side chain. In one embodiment, the amino acid residue found at position 66 in the F₀ sequence shown in SEQ ID NO:2 is substituted with Glutamic Acid (E). While not wishing to be bound by theory, it is believed that the change in charge polarity at amino acid 66 may alter the ability of F to bind to cell surface receptors, thereby influencing syncytium formation and spread of the virus. Alternately, the charge of the amino acid at position 66 may affect local intra- and/or inter-molecular electrostatic interactions and, in turn, the ability of the pre-fusion conformation to be triggered.

In particular, recombinant attenuated virus with a positive side chain at position 66, such as Lysine, in the RSV F subunit has been observed to grow to high titers in Vero and serum-free Vero cell culture and demonstrate efficient fusogenicity. In contrast, recombinant attenuated virus with a negatively charged side chain at position 66, such as glutamic acid, in the RSV F subunit has been observed to grow to lower titers in Vero and serum-free Vero cells and demonstrate reduced fusogenicity. However, in non-attenuated RSV virus, changing the amino acid residue at position 66 of the F subunit shown in SEQ ID NO:2 from Lysine (K) to Glutamic Acid (E), does not significantly affect viral growth.

In one embodiment, the amino acid at position 66 of F₀ sequence is an amino acid with a positive side chain selected from Arginine (R) or histidine (H). In one embodiment, the Lysine at position 66 of F₀ in SEQ ID NO:2 is substituted with Arginine or Histidine and can be abbreviated K66R or K66H. Nucleic acids encoding recombinant RSV with an F subunit having one or more mutations described above are also provided. In one embodiment, the nucleic acid is DNA, for example, cDNA. In another embodiment, the nucleic acid is RNA, for example, mRNA. In one embodiment, the nucleic acid is included within a vector, for example, a plasmid.

In one embodiment, recombinant RSV includes both a mutation in M2-2, as described above, and a substitution at residue 66 of the F subunit, as described above. In one embodiment, recombinant RSV that exhibits an attenuated phenotype is provided, wherein recombinant RSV includes one or more artificially altered amino acids, for example, at least one deleted, inserted and/or substituted amino acid that inactivates the M2-2 gene product and/or ablate expression of the M2-2 gene and an F subunit having at least one artificially mutated amino acid, for example, at least one deleted, inserted and/or substituted amino acid. In one embodiment, recombinant RSV includes a mutation in which at least a part of the M2-2 protein is deleted and an F subunit in which at least one amino acid is substituted.

In a more particular embodiment, recombinant RSV includes a mutation in which at least about 5, 10, 15, 20, 25, 30, 35, 40, or 45 amino acid residues from an amino acid sequence of an M2-2 protein shown in SEQ ID NO:4 and up to at least about 50, 55, 60, 65, 70, 75, 80, 85, or 90 amino acid residues from an amino acid sequence of an M2-2 protein shown in SEQ ID NO: 4 are deleted, wherein the deletion is sufficient to render the M2-2 protein inactive and/or prevent expression of the M2-2 protein and wherein a naturally occurring amino acid found at position 66 of a F subunit shown in SEQ ID NO:2 is substituted with an amino acid having a negative side chain. In one embodiment, recombinant RSV has an amino acid sequence that is at least about 75%, 80%, 85%, 90%, 95%, 96%, 97%, 98%, or 99% identical to an amino acid sequence of an M2-2 protein shown in SEQ ID NO: 4 and includes a mutation in which at least about 5, 10, 15, 20, 25, 30, 35, 40, or 45 amino acid residues from an amino acid sequence of an M2-2 protein shown in SEQ ID NO:4 and up to at least about 50, 55, 60, 65, 70, 75, 80, 85, or 90 amino acid residues from an amino acid sequence of an M2-2 protein shown in SEQ ID NO: 4 are deleted, wherein the deletion is sufficient to render the M2-2 protein inactive and/or prevent expression of the M2-2 protein and wherein the recombinant RSV includes a F subunit having an amino acid sequence that is at least about 75%, 80%, 85%, 90%, 95%, 96%, 97%, 98%, or 99% identical to an amino acid sequence of a F subunit shown in SEQ ID NO: 2, wherein a naturally occurring amino acid found at position 66 of the F subunit shown in SEQ ID NO:2 is substituted with an amino acid having a negative side chain. In one embodiment, one or more amino acids are deleted from the N-terminus of the amino acid sequence of M2-2 shown in SEQ ID NO:4. In one embodiment, one or more amino acids are deleted from the C-terminus of the amino acid sequence of M2-2 shown in SEQ ID NO:4.

In one embodiment, recombinant RSV includes a deletion of at least about 5%, 10%, 15%, 20%, 25%, 30%, 35%, 40%, 45% or 50% of the amino acid residues of an amino acid sequence of an M2-2 protein shown in SEQ ID NO:4 and up to at least about 55%, 60%, 65%, 70%, 75%, 80%, 85%, 90%, 95% or 100% of the amino acid residues of an amino acid sequence of an M2-2 protein shown in SEQ ID NO:4, wherein the deletion is sufficient to render the M2-2 protein inactive and/or prevent expression of the M2-2 protein and wherein a naturally occurring amino acid found at position 66 of an amino acid sequence of an F subunit shown in SEQ ID NO: 2 is substituted with an amino acid having a negative side chain. In one embodiment, recombinant RSV has an amino acid sequence that is at least about 75%, 80%, 85%, 90%, 95%, 96%, 97%, 98%, or 99% identical to an amino acid sequence of an M2-2 protein shown in SEQ ID NO: 4 and includes a mutation in which at least about 5%, 10%, 15%, 20%, 25%, 30%, 35%, 40%, 45% or 50% of the amino acid residues of an amino acid sequence of an M2-2 protein shown in SEQ ID NO:4 and up to at least about 55%, 60%, 65%, 70%, 75%, 80%, 85%, 90%, 95% or 100% of the amino acid residues of an amino acid sequence of an M2-2 protein shown in SEQ ID NO:4 are deleted, wherein the deletion is sufficient to render the M2-2 protein inactive and/or prevent expression of the M2-2 protein and wherein the recombinant RSV includes a F subunit having an amino acid sequence that is at least about 75%, 80%, 85%, 90%, 95%, 96%, 97%, 98%, or 99% identical to an amino acid sequence of a F subunit shown in SEQ ID NO: 2, wherein a naturally occurring amino acid found at position 66 of an amino acid sequence of an F subunit shown in SEQ ID NO: 2 is substituted with an amino acid having a negative side chain. In one embodiment, one or more amino acids are deleted from the N-terminus of the amino acid sequence of M2-2 shown in SEQ ID NO:4. In one embodiment, one or more amino acids are deleted from the C-terminus of the amino acid sequence of M2-2 shown in SEQ ID NO:4.

In one embodiment, recombinant RSV F subunit does not include a negatively charged amino acid such as Glutamic Acid (E) at residue 66. In another embodiment, the amino acid residue at position 66 is not Lysine (K). In a more particular embodiment, recombinant RSV includes a mutation in the F subunit position 66 in which amino acid having a positive side chain is selected from Arginine (R) or histidine (H). Nucleic acids encoding recombinant RSV with a mutation in M2-2 and the F subunit, as described above, are also provided. In one embodiment, the nucleic acid is DNA, for example, cDNA. In another embodiment, the nucleic acid is RNA, for example, mRNA. In one embodiment, the nucleic acid is included within a vector, for example, a plasmid.

### 6. Vaccines

In another embodiment, immunogenic compositions that include an immunologically effective amount of a recombinant respiratory syncytial virus, polypeptide, and/or nucleic acid are provided. In one embodiment, the immunogenic composition includes an immunologically effective amount of a respiratory syncytial virus, polypeptide, and/or nucleic acid in a physiologically acceptable carrier.

In one embodiment, the immunogenic composition is an RSV vaccine, for example, a live attenuated RSV vaccine. In one embodiment, the vaccine includes an immunologically effective amount of recombinant RSV having an attenuated phenotype as described herein. In one embodiment, the vaccine includes an immunologically effective amount of recombinant RSV in which one or more amino acids have been artificially altered, for example, in which at least one amino acid has been deleted, inserted and/or substituted. In one embodiment, the vaccine includes an immunologically effective amount of recombinant RSV having one or more mutations that inactivate the M2-2 gene product and/or ablate expression of the M2-2 gene. In one embodiment, the vaccine includes an immunologically effective amount of recombinant RSV having a mutation in which at least a part of the M2-2 protein is deleted, as described in detail above. In one embodiment, one or more amino acids are deleted from the N-terminus of M2-2. In one embodiment, one or more amino acids are deleted from the C-terminus of M2-2. In one embodiment, the vaccine includes an immunologically effective amount of recombinant RSV which includes an F subunit having at least one artificially mutated amino acid, for example, at least one deleted, inserted and/or substituted amino acid. In one embodiment, the vaccine includes an immunologically effective amount of recombinant RSV that includes an F subunit having at least one substituted amino acid. In a more particular embodiment, the vaccine includes an immunologically effective amount of recombinant RSV that includes an F subunit in which a naturally occurring amino acid found at position 66 of an amino acid sequence of an F subunit shown in SEQ ID NO:2 is artificially substituted with an amino acid residue having a negative side chain. In one embodiment, the vaccine includes an immunologically effective amount of recombinant RSV wherein the F subunit includes a negatively charged amino acid such as Glutamic Acid (E) at residue 66. In another embodiment, the amino acid residue at position 66 is not Lysine (K). In one embodiment, recombinant RSV F subunit does not include a negatively charged amino acid such as Glutamic Acid (E) at residue 66. In one embodiment, recombinant RSV includes a mutation in the F subunit position 66 in which amino acid having a positive side chain is selected from Arginine (R) or histidine (H). In one embodiment, the vaccine includes an immunologically effective amount of recombinant RSV in which the Lysine found at position 66 in the amino acid sequence of the F subunit shown in SEQ ID NO:2 is artificially substituted with an amino acid having a negative side chain.

In one embodiment, the vaccine includes an immunologically effective amount of recombinant RSV that includes both a mutation in M2-2, as described above, and a substitution at residue 66 of the F subunit as described above. In one embodiment, the vaccine includes an immunologically effective amount of recombinant RSV that exhibits an attenuated phenotype, wherein recombinant RSV includes one or more artificially altered amino acids, for example, at least one deleted, inserted and/or substituted amino acid that inactivate the M2-2 gene product and/or ablate expression of the M2-2 gene and an F subunit having at least one mutated amino acid, for example, at least one deleted, inserted and/or substituted amino acid. In one embodiment, the vaccine includes an immunologically effective amount of recombinant RSV that includes a mutation in which at least a part of the M2-2 protein is deleted and having an F subunit with at least one substituted amino acid.

In a more particular embodiment, the vaccine includes an immunologically effective amount of recombinant RSV having a mutation in which at least about 5, 10, 15, 20, 25, 30, 35, 40, or 45 amino acid residues from an amino acid sequence of a M2-2 protein shown in SEQ ID NO:4 and up to at least about 50, 55, 60, 65, 70, 75, 80, 85, or 90 amino acid residues from an amino acid sequence of a M2-2 protein shown in SEQ ID NO: 4 are deleted, wherein the deletion is sufficient to render the M2-2 protein inactive and/or prevent expression of the M2-2 protein and wherein a naturally occurring amino acid found at position 66 of an amino acid sequence of an F subunit shown in SEQ ID NO: 2 is substituted with an amino acid having a negative side chain. In a more particular embodiment, the vaccine includes an immunologically effective amount of recombinant RSV having an amino acid sequence that is at least about 75%, 80%, 85%, 90%, 95%, 96%, 97%, 98%, or 99% identical to an amino acid sequence of an M2-2 protein shown in SEQ ID NO: 4 and includes a mutation in which at least about 5, 10, 15, 20, 25, 30, 35, 40, or 45 amino acid residues from an amino acid sequence of a M2-2 protein shown in SEQ ID NO:4 and up to at least about 50, 55, 60, 65, 70, 75, 80, 85, or 90 amino acid residues from an amino acid sequence of a M2-2 protein shown in SEQ ID NO: 4 are deleted, wherein the deletion is sufficient to render the M2-2 protein inactive and/or prevent expression of the M2-2 protein and wherein the recombinant RSV includes a F subunit having an amino acid sequence that is at least about 75%, 80%, 85%, 90%, 95%, 96%, 97%, 98%, or 99% identical to an amino acid sequence of a F subunit shown in SEQ ID NO: 2, and wherein a naturally occurring amino acid found at position 66 of an amino acid sequence of an F subunit shown in SEQ ID NO: 2 is substituted with an amino acid having a negative side chain. In one embodiment, one or more amino acids are deleted from the N-terminus of the amino acid sequence of M2-2 shown in SEQ ID NO:4. In one embodiment, one or more amino acids are deleted from the C-terminus of the amino acid sequence of M2-2 shown in SEQ ID NO:4.

In one embodiment, the vaccine includes an immunologically effective amount of recombinant RSV that includes a deletion of at least about 5%, 10%, 15%, 20%, 25%, 30%, 35%, 40%, 45% or 50% of the amino acid residues of an amino acid sequence of a M2-2 protein shown in SEQ ID NO:4 and up to at least about 55%, 60%, 65%, 70%, 75%, 80%, 85%, 90%, 95% or 100% of the amino acid residues of an amino acid sequence of a M2-2 protein shown in SEQ ID NO:4, wherein the deletion is sufficient to render the M2-2 protein inactive and/or prevent expression of the M2-2 protein and wherein a naturally occurring amino acid found at position 66 of an amino acid sequence of an F subunit shown in SEQ ID NO: 2 is substituted with an amino acid having a negative side chain. In a more particular embodiment, the vaccine includes an immunologically effective amount of recombinant RSV having an amino acid sequence that is at least about 75%, 80%, 85%, 90%, 95%, 96%, 97%, 98%, or 99% identical to an amino acid sequence of an M2-2 protein shown in SEQ ID NO: 4 and includes a deletion of at least about 5%, 10%, 15%, 20%, 25%, 30%, 35%, 40%, 45% or 50% of the amino acid residues of an amino acid sequence of a M2-2 protein shown in SEQ ID NO:4 and up to at least about 55%, 60%, 65%, 70%, 75%, 80%, 85%, 90%, 95% or 100% of the amino acid residues of an amino acid sequence of a M2-2 protein shown in SEQ ID NO:4, wherein the deletion is sufficient to render the M2-2 protein inactive and/or prevent expression of the M2-2 protein and wherein the recombinant RSV includes a F subunit having an amino acid sequence that is at least about 75%, 80%, 85%, 90%, 95%, 96%, 97%, 98%, or 99% identical to an amino acid sequence of a F subunit shown in SEQ ID NO: 2, and wherein a naturally occurring amino acid found at position 66 of an amino acid sequence of an F subunit shown in SEQ ID NO: 2 is substituted with an amino acid having a negative side chain. In one embodiment, one or more amino acids are deleted from the N-terminus of the amino acid sequence of M2-2 shown in SEQ ID NO:4. In one embodiment, one or more amino acids are deleted from the C-terminus of the amino acid sequence of M2-2 shown in SEQ ID NO:4.

In one embodiment, the vaccine includes an immunologically effective amount of recombinant RSV in which the F subunit includes a negatively charged amino acid such as Glutamic Acid (E) at residue 66. In another embodiment, the vaccine includes an immunologically effective amount of recombinant RSV in which the amino acid residue at position 66 is not Lysine (K). In one embodiment, the vaccine includes a physiologically acceptable carrier and/or adjuvant.

### 7. Recombinant Expression

In one embodiment, the vaccine composition includes RSV having an attenuated phenotype. In one embodiment, the vaccine composition includes recombinantly produced RSV. In a more particular embodiment, the vaccine composition includes recombinantly produced RSV having either a deletion in the M2-2 protein, as described above, a mutation in an F subunit, as described previously, or a combination thereof.

To recombinantly produce RSV, an open reading frame (ORF) encoding the protein may be inserted or cloned into a vector for replication of the vector, transcription of a portion of the vector (e.g., transcription of the ORF) and/or expression of the protein in a cell. The term “open reading frame” (ORF) refers to a nucleic acid sequence that encodes a protein that is located between a start codon (AUG in ribonucleic acids and ATG in deoxyribonucleic acids) and a stop codon (e.g., UAA (ochre), UAG (amber) or UGA (opal) in ribonucleic acids and TAA, TAG or TGA in deoxyribonucleic acids). A vector may also include elements that facilitate cloning of the ORF or other nucleic acid element, replication, transcription, translation and/or selection. Thus, a vector may include one or more or all of the following elements: one or more promoter elements, one or more 5′ untranslated regions (5′UTRs), one or more regions into which a target nucleotide sequence may be inserted (an “insertion element”), one or more ORFs, one or more 3′ untranslated regions (3′UTRs), and a selection element. Any convenient cloning strategy known in the art may be used to incorporate an element, such as an ORF, into a vector nucleic acid.

In one embodiment, reverse genetics is used to introduce one or more mutations in the genome of a negative stranded RNA virus such as RSV. In reverse genetics, the viral genome is first reverse transcribed into a cDNA clone, which can be manipulated, for example, by the introduction of one or more mutations. To create an infectious, recombinant RNA virus, the cDNA clone is “rescued” or converted back into RNA. The nucleotide sequence of a cDNA clone that includes the RSV genome and can be used to rescue recombinant RSV, for example, rA2ΔM2-2, is shown in SEQ ID NO: 5. In one embodiment, the cDNA clone used to rescue recombinant RSV has a sequence that is at least 75%, 80%, 85%, 90%, 95%, 96%, 97%, 98%, or 99% identical to the sequence shown in SEQ ID NO:5. A nucleotide sequence of pUC19+rA2ΔM2-2 plasmid, used to rescue recombinant virus is shown in SEQ ID NO:6. In one embodiment, the plasmid used to rescue recombinant RSV has a sequence that is at least 75%, 80%, 85%, 90%, 95%, 96%, 97%, 98%, or 99% identical to the sequence shown in SEQ ID NO:6.

Various types of mutagenesis can be used to modify nucleic acids and encoded polypeptides and/or viruses to produce conservative or non-conservative variants. Mutagenesis procedures optionally include selection of mutant nucleic acids and polypeptides for one or more activity of interest. Procedures that can be used include, but are not limited to: site-directed point mutagenesis, random point mutagenesis, in vitro or in vivo homologous recombination (DNA shuffling), mutagenesis using uracil containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, double-strand break repair, and many others known to persons of skill Mutagenesis, e.g., involving chimeric constructs, can also be used. In one embodiment, mutagenesis can be guided by known information of the naturally occurring molecule or altered or mutated naturally occurring molecule, e.g., sequence, sequence comparisons, physical properties, crystal structure or the like.

Detailed protocols for manipulation of viral nucleic acids and/or proteins, including amplification, cloning, mutagenesis, transformation, and the like, are described in, e.g., in Ausubel et al. Current Protocols in Molecular Biology (supplemented through 2003) John Wiley & Sons, New York (“Ausubel”); Sambrook et al. Molecular Cloning—A Laboratory Manual (3rd Ed.), Vol. 1-3, Cold Spring Harbor Laboratory, Cold Spring Harbor, N.Y., 2001 (“Sambrook”), and Berger and Kimmel Guide to Molecular Cloning Techniques, Methods in Enzymology volume 152 Academic Press, Inc., San Diego, Calif. (“Berger”), the disclosures of which are hereby incorporated by reference herein in their entirety.

### 8. Cell Culture

Typically, propagation of a recombinant virus (e.g., recombinant RSV) is accomplished in media compositions in which the host cell is commonly cultured. Suitable host cells for the replication of RSV include Vero cells and HEp-2 cells. Typically, cells are cultured in a standard commercial culture medium, such as Dulbecco's modified Eagle's medium supplemented with serum (e.g., 10% fetal bovine serum), or in serum free medium, under controlled humidity and CO₂ concentration suitable for maintaining neutral buffered pH (e.g., at pH between 7.0 and 7.2). Optionally, the medium contains antibiotics to prevent bacterial growth, e.g., penicillin, streptomycin, etc., and/or additional nutrients, such as L-glutamine, sodium pyruvate, non-essential amino acids, additional supplements to promote favorable growth characteristics, e.g., trypsin, β-mercaptoethanol, and the like.

Procedures for maintaining mammalian cells in culture have been extensively reported, and are known to those of skill in the art. General protocols are provided, e.g., in Freshney (1983) Culture of Animal Cells: Manual of Basic Technique, Alan R. Liss, New York; Paul (1975) Cell and Tissue Culture, 5.sup.th ed., Livingston, Edinburgh; Adams (1980) Laboratory Techniques in Biochemistry and Molecular Biology—Cell Culture for Biochemists, Work and Burdon (eds.) Elsevier, Amsterdam, the disclosures of which are hereby incorporated by references herein in their entirety. Variations in such procedures are also possible.

Cells for production of RSV can be cultured in serum-containing or serum free medium. In some cases, e.g., for the preparation of purified viruses, it may be desirable to grow the host cells in serum free conditions. Cells can be cultured in small scale, e.g., less than 25 ml medium, culture tubes or flasks or in large flasks with agitation, in rotator bottles, or on microcarrier beads (e.g., DEAE-Dextran microcarrier beads, such as Dormacell, Pfeifer & Langen; Superbead, Flow Laboratories; styrene copolymer-tri-methylamine beads, such as Hillex, SoloHill, Ann Arbor) in flasks, bottles or reactor cultures. Microcarrier beads are small spheres (in the range of 100-200 microns in diameter) that provide a large surface area for adherent cell growth per volume of cell culture. For example a single liter of medium can include more than 20 million microcarrier beads providing greater than 8000 square centimeters of growth surface. For commercial production of viruses, e.g., for vaccine production, it is often desirable to culture the cells in a bioreactor or fermenter. Bioreactors are available in volumes from under 1 liter to in excess of 100 liters, e.g., Cyto3 Bioreactor (Osmonics, Minnetonka, Minn.); NBS bioreactors (New Brunswick Scientific, Edison, N.J.); laboratory and commercial scale bioreactors from B. Braun Biotech International (B. Braun Biotech, Melsungen, Germany).

### 9. Introduction of Vectors Into Host Cells

Vectors incorporating polynucleotides encoding RSV can be are introduced into host cells according to methods well known in the art for introducing heterologous nucleic acids into eukaryotic cells, including, for example, calcium phosphate co-precipitation, electroporation, microinjection, lipofection, and transfection employing polyamine transfection reagents. For example, vectors such as plasmids, can be transfected into host cells using the transfection reagent LipofectACE or Lipofectamine 2000 (Invitrogen) according to the manufacturer's instructions. Alternatively, electroporation can be employed to introduce vectors incorporating RSV genome segments into host cells.

### 10. Methods of Use

In another embodiment, methods for stimulating the immune system of a mammal to produce an immune response against RSV are provided. In one embodiment, the immune response is a protective immune response. In one embodiment, the immune response is humoral. In another embodiment, the immune response is cell-mediated. In one embodiment, the method induces a protective immune response to RSV infection or at least one symptom thereof. Also included are methods for preventing or treating a disease by administering to a patient having said disease, or at risk of contracting said disease, a therapeutically, or prophylactically, effective amount of the vaccine composition. In one embodiment, the disease is a disease of the respiratory system, for example, a disease is caused by a virus, in particular RSV. In one embodiment, a method of inducing neutralizing antibodies against RSV in a mammal is provided. In one embodiment, administration of the vaccine composition results in a reduction in RSV viral titers.

In one embodiment, the method includes administering to a mammal recombinant RSV having an attenuated phenotype. In one embodiment, the mammal is a human. In one embodiment, the method includes administering to a mammal an immunologically effective amount of recombinant RSV having an attenuated phenotype as described herein. In one embodiment, the method includes administering an immunologically effective amount of recombinant RSV in which one or more amino acids have been artificially altered, for example, in which at least one amino acid has been deleted, inserted and/or substituted. In one embodiment, the method includes administering an immunologically effective amount of recombinant RSV having one or more mutations that inactivate the M2-2 gene product and/or ablate expression of the M2-2 gene. In one embodiment, the method includes administering an immunologically effective amount of recombinant RSV having a mutation in which at least a part of the M2-2 protein is deleted, as described in detail above. In one embodiment, one or more amino acids are deleted from the N-terminus of M2-2. In one embodiment, one or more amino acids are deleted from the C-terminus of M2-2.

In one embodiment, the method includes administering an immunologically effective amount of recombinant RSV which includes an F subunit having at least one mutated amino acid, for example, at least one deleted, inserted and/or substituted amino acid. In one embodiment, the method includes administering an immunologically effective amount of recombinant RSV which includes an F subunit having at least one artificially mutated amino acid, for example, at least one deleted, inserted and/or substituted amino acid. In one embodiment, the method includes administering an immunologically effective amount of recombinant RSV that includes an F subunit having at least one substituted amino acid. In a more particular embodiment, the method includes administering an immunologically effective amount of recombinant RSV that includes an F subunit having at least one artificially mutated amino acid residue at position 66. In one embodiment, the method includes administering an immunologically effective amount of recombinant RSV wherein the F subunit includes a negatively charged amino acid such as Glutamic Acid (E) at residue 66. In one embodiment, the amino acid residue at position 66 is not Glutamic Acid (E). In another embodiment, the amino acid residue at position 66 is not Lysine (K). In one embodiment, the method includes administering an immunologically effective amount of recombinant RSV in which the Lysine found at position 66 in the amino acid sequence of the F subunit shown in SEQ ID NO:2 is artificially with an amino acid having a negative side chain. In one embodiment, the amino acid with a negative side chain is Glutamic Acid (E). In one embodiment, the method includes administering an immunologically effective amount of recombinant RSV that includes both a mutation in M2-2, as described above, and a substitution at residue 66 of the F subunit, as described above.

Recombinant RSV can be administered in an appropriate carrier or excipient. Typically, the carrier or excipient is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions insuring sterility, pH, isotonicity, and stability is effected according to protocols established in the art. Generally, a carrier or excipient is selected to minimize allergic and other undesirable effects, and to suit the particular route of administration, such as subcutaneous, intramuscular, intranasal, oral, topical, etc. The resulting aqueous solutions can be packaged for use as a liquid or lyophilized, wherein the lyophilized preparation is combined with a sterile solution prior to administration

Dosages and methods for eliciting a protective anti-viral immune response, adaptable to producing a protective immune response against RSV are known to those of skill in the art. Typically, the dose will be adjusted based on patient characteristics such as age, physical condition, body weight, sex, diet, other factors such as mode and time of administration, and other clinical factors. In one embodiment, recombinant RSV is provided in the range of about 10³-10⁶ pfu (plaque forming units) per dose administered (e.g., 10⁴-10⁵ pfu per dose administered). The vaccine formulation can be systemically administered by subcutaneous or intramuscular injection using a needle and syringe or a needleless injection device. In one embodiment, the vaccine formulation is administered intranasally, for example, using a spray, drops, or aerosol into the upper respiratory tract (e.g., the nasopharynx). While any of the above routes of delivery results in a protective systemic immune response, intranasal administration confers the added benefit of eliciting mucosal immunity at the site of entry of the virus.

In one embodiment, a protective immune response is elicited with a single dose. In other embodiments, more than one dose is administered to achieve the desired level of protection. Additional doses can be administered by the same or different route. In neonates and infants, for example, multiple administrations may be required to elicit sufficient levels of immunity. Administration can continue at intervals throughout childhood, as necessary to maintain sufficient levels of protection against wild-type RSV infection. Similarly, adults who are particularly susceptible to repeated or serious RSV infection, such as, for example, health care workers, day care workers, family members of young children, elderly, mammals with compromised cardiopulmonary function, etc. may require multiple immunizations to establish and/or maintain protective immune responses. Levels of induced immunity can be monitored, for example, by measuring amounts of neutralizing secretory and serum antibodies, and dosages adjusted or vaccinations repeated as necessary to maintain desired levels of protection.

Alternatively, an immune response can be stimulated by ex vivo or in vivo targeting of dendritic cells with virus. For example, proliferating dendritic cells can be exposed to recombinant RSV in a sufficient amount and for a sufficient period of time to permit capture of the RSV antigens by the dendritic cells. The cells are then transferred into a subject to be vaccinated by standard intravenous transplantation methods.

In one embodiment, the formulation contains one or more adjuvants for enhancing the immune response to the RSV antigens. Suitable adjuvants include, for example: complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, bacille Calmette-Guerin (BCG), Corynebacterium parvum, and the synthetic adjuvant QS-21.

In one embodiment, recombinant RSV is administered in conjunction with one or more immunostimulatory molecules. Immunostimulatory molecules include various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, F1t3 ligand, B7.1; B7.2, etc. The immunostimulatory molecules can be administered in the same formulation as the RSV, or can be administered separately.

### 11. Kits

In one embodiment, recombinant RSV as described herein and, optionally, additional components, such as, buffer, cells, and culture medium, useful for producing recombinant RSV, can be packaged in the form of a kit. In one embodiment, the kit includes instructions for performing the methods, packaging material, and/or one or more containers.

In one embodiment a pharmaceutical pack or kit that includes one or more containers filled with one or more of the ingredients of the vaccine formulations is provided. The vaccine composition can be packaged in a hermetically sealed container such as an ampoule or sachette indicating the quantity of composition. In one embodiment, the composition is supplied as a liquid. In another embodiment, the composition is supplied as a dry sterilized lyophilized powder or water free concentrate in a hermetically sealed container, wherein the composition can be reconstituted, for example, with water or saline, to obtain an appropriate concentration for administration to a subject.

When the vaccine composition is systemically administered, for example, by subcutaneous or intramuscular injection, a needle and syringe, or a needle-less injection device can be used. The vaccine formulation can be enclosed in ampoules, disposable syringes or multiple dose vials made of glass or plastic.

### INCORPORATION BY REFERENCE

All references cited herein, including patents, patent applications, papers, text books and the like, and the references cited therein, to the extent that they are not already, are hereby incorporated herein by reference in their entirety.

### WORKING EXAMPLES

Sequence Information:

| SEQ ID NO: | Description |
|---|---|
| 1 | nucleotide sequence of F₀ from RSV strain A2 |
| 2 | amino acid sequence of F₀ from RSV strain A2 |
| 3 | nucleotide sequence of M2-2 from RSV strain A2 |
| 4 | amino acid sequence of M2-2 from RSV strain A2 |
| 5 | nucleotide sequence of rA2ΔM2-2 cDNA |
| 6 | nucleotide sequence of pUC19+ rA2ΔM2-2 plasmid |
| | (used in rescue of recombinant virus) |

The reagents employed in the examples are commercially available or can be prepared using commercially available instrumentation, methods, or reagents known in the art. The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

### A. Introduction

rA2ΔM2-2(NIH) and rA2ΔM2-2(MEDI) are two RSV vaccines that are attenuated by deletion of the M2-2 open reading frame. Though both rA2ΔM2-2 viruses are derived from RSV A2, they carry 4 amino acid differences and have different deletions of the M2-2 gene. The two versions of rA2ΔM2-2, were evaluated in serum-free (SF) adapted Vero cells (Yuk et al., 2006, Cytotechnology, 51:183-192; Tang et al., 2008, J Virol Methods, 153: 196-202). In this SF Vero cell line, the two versions of rA2ΔM2-2 showed different growth kinetics and cytopathic effect (CPE)—rA2ΔM2-2(MEDI) grew to 100-fold higher titers than rA2ΔM2-2(NIH) with markedly larger syncytia.

The growth differences were unexpected because both versions are derived from the A2 strain of RSV and share >99% sequence identity. Alignment of their genome sequences identified four predicted amino acid differences located in three viral proteins: NS2, N and F. Each of the different amino acids found in rA2ΔM2-2(NIH) was introduced into rA2ΔM2-2(MEDI) in order to assess its effect on growth. The amino acid at position 66 in the F₂ fragment of the RSV fusion protein was identified as the genetic determinant of the observed growth differences between the two rA2ΔM2-2 viruses. Transfection experiments involving substitution by amino acids with different groups of chemical properties at this position in RSV F further demonstrated that basic amino acids with positively charged side chains resulted in efficient fusion activity, whereas negatively charged amino acids reduced fusion activity.

B. Differences in Growth Between Two rA2ΔM2-2 Vaccine Candidates

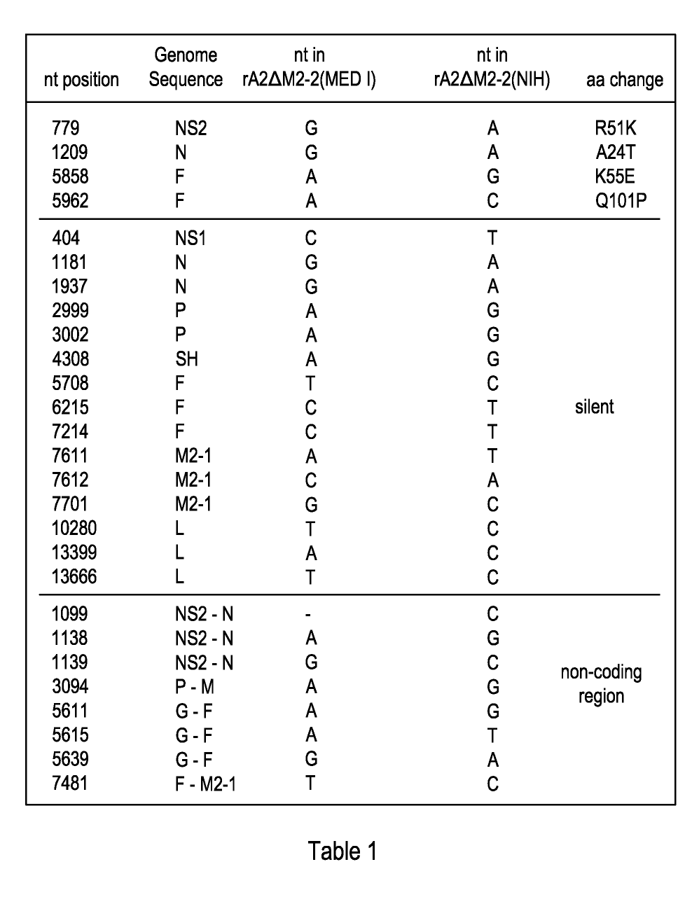

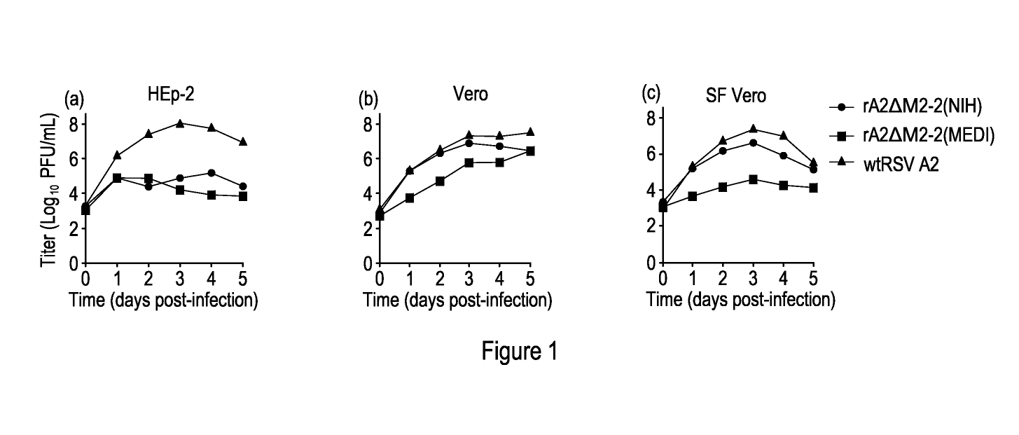

Both versions of rA2ΔM2-2 have reduced growth in various cell lines as well as attenuated growth in rodents and non-human primates (Teng et al., 2000, J. Virol. 74:9317-9321; Jin et al., 2000, J. Virol. 74:74-82). Multi-cycle growth curve analysis of rA2ΔM2-2(MEDI) and rA2ΔM2-2(NIH) in three different cell lines is shown in FIG. 1. In HEp-2 cells, both rA2ΔM2-2 viruses grew poorly, as previously reported, with peak titers >100-fold lower than titers of wt RSVA2 (FIG. 1a). Next we compared growth in both a Vero cell line obtained from ATCC as well as a serum-free (SF) adapted Vero cell line (Yuk et al., 2006, Cytotechnology, 51:183-192). In the parental Vero cell line, rA2ΔM2-2(MEDI) had faster growth kinetics than rA2ΔM2-2(NIH) (FIG. 1b). On day 2 post infection (p.i.), rA2ΔM2-2(MEDI) had titer of 6.3 log₁₀ PFU/ml and rA2ΔM2-2(NIH) had only 4.7 log₁₀ PFU/ml, though both rA2ΔM2-2(MEDI) and rA2ΔM2-2(NIH) reached a titer of 6.5 log₁₀ PFU/ml on day 5. The difference in growth kinetics was even more evident in the SF Vero cell line, where rA2ΔM2-2(MEDI) had 100-fold higher titer than rA2ΔM2-2(NIH) by day 2 p.i. (FIG. 1c). In SF Vero cells, rA2ΔM2-2(MEDI) reached a peak titer of 6.6 log₁₀ PFU/ml while rA2ΔM2-2(NIH) reached a peak titer of only 4.6 log₁₀ PFU/ml.

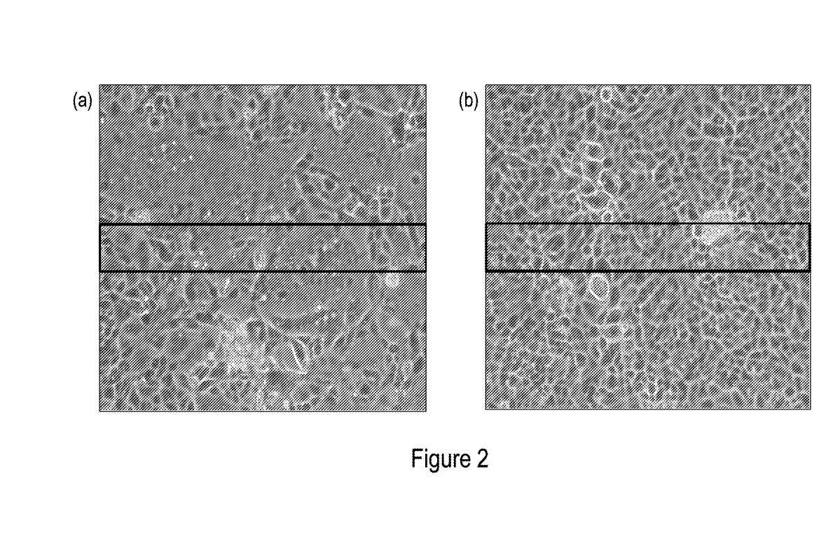

In addition to the difference in growth kinetics, these viruses showed marked differences in cytopathic effect (CPE). Vero cells infected with rA2ΔM2-2(MEDI) generated large syncytia over the entire cell monolayer by 48 h.p.i. (FIG. 2a). In contrast, the rA2ΔM2-2(NIH) virus had only small syncytia that were associated with phase-bright cell clusters (FIG. 2b). Similar differences in CPE were observed in the SF Vero cell line. These results show that the rA2ΔM2-2(MEDI) virus has faster growth kinetics and generates larger syncytia in Vero cells compared to rA2ΔM2-2(NIH).

### C. Identification of K66E as the Major Genetic Determinant for Altered Growth

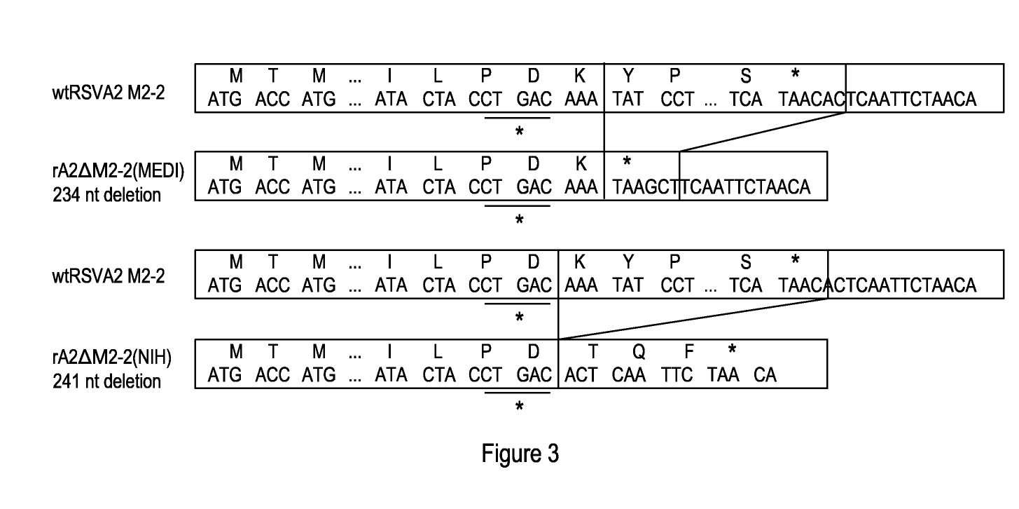

Though both rA2ΔM2-2(MEDI) and rA2ΔM2-2(NIH) have a deletion in the M2-2 open reading frame and are derived from strain RSV A2, there are differences in the M2-2 deletion as well as in their genomic sequences. In order to identify the genetic determinants responsible for the growth differences between these two viruses we performed an alignment of their cDNA sequences. The results of the alignment identified 34 nucleotide differences: 4 differences encoding amino acid (aa) changes in the NS2, N and F genes; 15 differences in coding regions that did not alter amino acid sequence; 8 differences in the non-coding regions and differences in the M2-2 deletion (Table 1 and FIG. 3).

Only the four nucleotide differences that encode amino acid changes were individually introduced into the rA2ΔM2-2(MEDI) cDNA. A fifth cDNA was generated in which the M2-2 gene deletion in rA2ΔM2-2(MEDI) was changed to mimic the analogous deletion in rA2ΔM2-2(NIH). Four recombinant virus variants each carrying one of the single amino acid changes (R51K in NS2, A24T in N, K66E in F and Q101P in F) and one virus carrying the rA2ΔM2-2(NIH) M2-2 deletion were generated from these cDNAs by reverse genetics for comparison of growth kinetics and CPE.

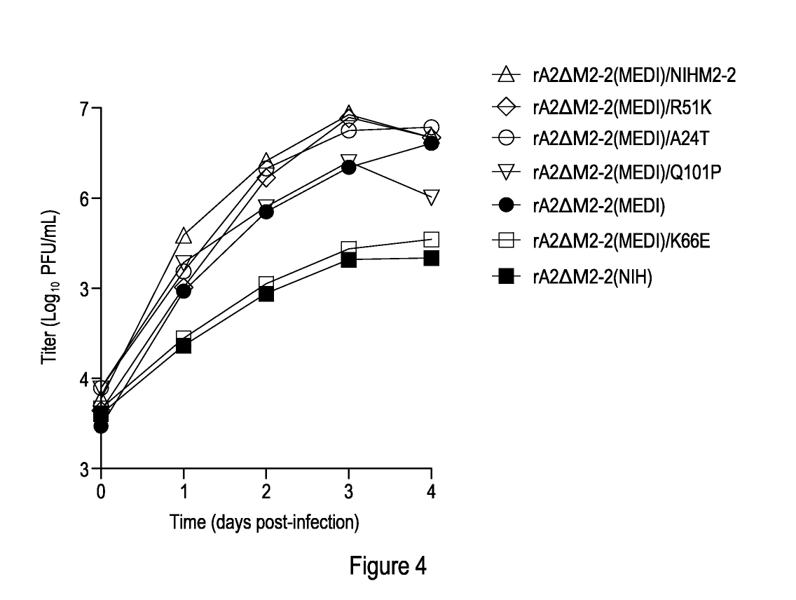

Both rA2ΔM2-2(NIH) and rA2ΔM2-2(MEDI)/K66E had similar growth kinetics with peak titers of only 5.3 and 5.5 log₁₀ PFU/ml, respectively (FIG. 4). The variant rA2ΔM2-2(MEDI)/K66E in Vero cells formed the same small syncytia and phase-bright cell clusters seen previously with rA2ΔM2-2(NIH). In contrast, the variants harboring R51K in NS2, A24T in N, and Q101P in F as well as the same deletion M2-2 deletion as rA2ΔM2-2(NIH) grew to peak titers similar to rA2ΔM2-2(MEDI) (FIG. 4). These results suggest that the K66E change in the F protein is the major genetic determinant for the reduced growth and altered CPE of rA2ΔM2-2(NIH) compared to rA2ΔM2-2(MEDI).

### D. A Change at Amino Acid 66 in RSV F Alters Fusion Activity

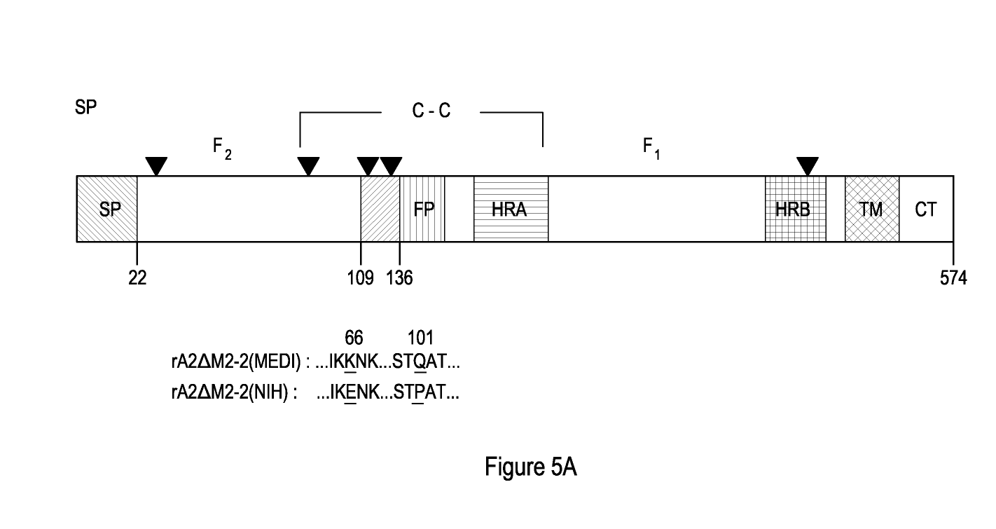

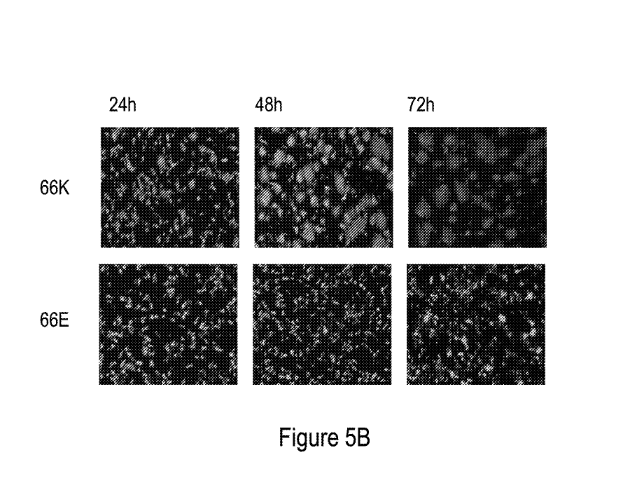

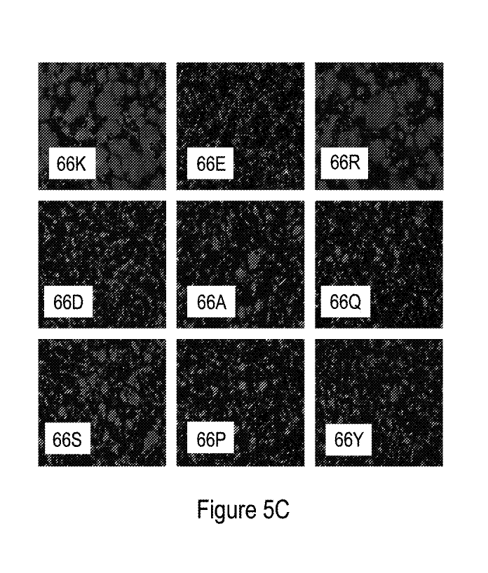

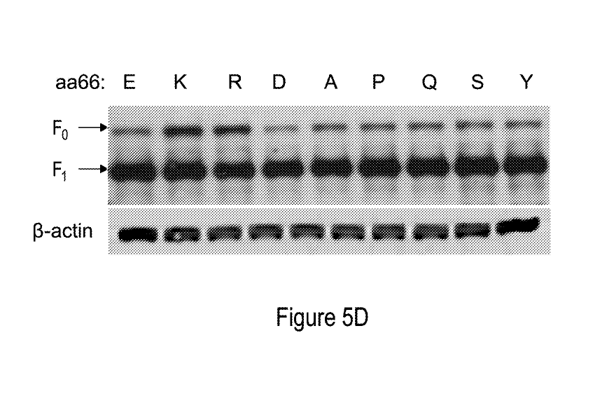

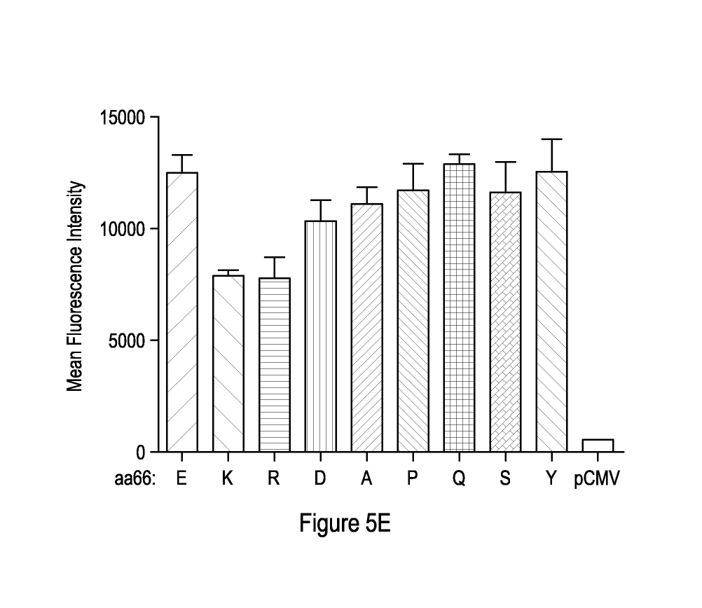

In order to analyze fusion activity of the RSV F protein outside the context of virus replication, a codon-optimized version of the RSV F gene was cloned into plasmid pCMV-Script. Transfection of Vero cells with this plasmid carrying a lysine at amino acid 66 in the RSV F gene (pF/66K) generated large syncytia by 72 h (FIG. 5b). In contrast, Vero cells transfected with the same plasmid carrying a glutamic acid residue (E) in the RSV F gene at amino acid 66 (pF/66E) formed only small syncytia (FIG. 5b). The differences in syncytium formation observed in Vero cells transfected with pF/66K and pF/66E recapitulated the differences seen in cells infected with the rA2ΔM2-2(MEDI) and rA2ΔM2-2(NIH) viruses, respectively. These results suggest that a single amino acid at position 66 in RSV F plays an important role in promoting fusion and confirm the altered growth of the rA2ΔM2-2(MEDI)/K66E virus.

### E. A Positive Charge at Amino Acid 66 is Required for Efficient Fusion Activity

To determine whether the polarity at position 66 is responsible for the differences in fusion, we generated pCMV/RSVF plasmids carrying either a positively charged arginine at position 66 (pF/66R) or a negatively charged aspartic acid at the same position (pF/66D). Transfection experiments showed that the RSV F mutant containing 66R produced large syncytia by 48 hr, whereas the RSV F mutant containing 66D produced small syncytia (FIG. 5c). Thus, amino acids lysine and arginine with positively charged side chains promoted efficient fusion, while amino acids glutamic acid and aspartic acid with negatively charged side chains hindered efficient fusion. To further test the influence of charge at position 66, RSV F plasmids containing substitutions with various amino acids carrying neutral side chains were similarly generated. Vero cell monolayers transfected with pF/66A, pF/66P, pF/66Q, pF/66S, or pF/66Y produced small to intermediate size syncytia (FIG. 5c). These results strongly suggest that electrostatic interactions at position 66 in the F₂ fragment of RSV F play a role in fusion.
RSV F protein is initially produced as a full length precursor (F₀) that is cleaved by a furin-like protease to form two disulfide-linked fragments (F₁ and F₂) of ˜50 kDa and ˜25 kDa, respectively. To confirm that the level of expression and proteolytic cleavage was equivalent among the different RSV F mutants, SDS-PAGE and western blotting was performed on lysates of transfected Vero cells. Blots probed with motavizumab to visualize F₀ and F₁ indicated that all the mutants had similar levels of RSV F expression and equivalent levels of processing at the furin cleavage site (FIG. 5d). Blots were re-probed with anti-β-actin to show equivalent amounts of protein loaded in each lane (FIG. 5d).

Since different levels of RSV F on the cell surface could also have an effect on syncytium formation, we compared cell surface expression levels of the various F mutants using flow cytometry. 293T cells were transfected with each plasmid, stained with motavizumab to detect cell surface RSV F, and subjected to FACS analysis. The data indicated that the two constructs that caused the most cell-to-cell fusion, pF/66K and pF/66R, actually had slightly less expression of RSV F on the cell surface as compared to cells transfected with the other F plasmids which caused markedly less fusion (FIG. 5e). These results suggest that the larger syncytia produced by pF/66K and pF/66R are not due to a higher amount of F protein on the cell surface, but are due to the ability of a positively charged residue at position 66 in F to facilitate fusion.

### F. Materials and Methods

i. Cell Lines and Virus

Vero cells (American Type Culture Collection (ATCC); not more than passage 148) were maintained in minimal essential medium (Gibco) supplemented with 5% heat-inactivated fetal bovine serum (FBS) (Hyclone), 2 mM L-glutamine (Invitrogen), and 100 U/ml penicillin with 100 μg/mL streptomycin (Invitrogen). Serum-free (SF) adapted Vero cells have been described previously (Yuk et al., 2006, Cytotechnology, 51:183-192) and were maintained in OptiPro SFM (Gibco) supplemented with 2 mM L-glutamine, and 100 U/ml penicillin with 100 μg/mL streptomycin. 293T cells (ATCC) were maintained in Dulbecco's minimal essential medium supplemented with 10% heat-inactivated FBS, 2 mM L-glutamine, and 100 U/ml penicillin with 100 μg/mL streptomycin. BSR/T7 cells (kindly provided by K. K. Conzelmann) were maintained in GMEM (Gibco) supplemented with 10% heat-inactivated FBS, 2% tryptone-phosphate broth (Sigma), and 100 μg/mL gentamycin (Gibco). All cell lines were cultured at 37° C. in 5% CO₂ incubators. wtRSVA2 virus was obtained from ATCC and passaged in Vero cells.

To propagate virus, Vero cells were infected at a multiplicity of infection (MOI)=0.01 PFU/cell in Optimem-I media (Gibco). When cytopathic effect (CPE) covered 70-80% of the monolayer, cells and supernatant were harvested together. Cryo-preservative (10× SP [2.18 M sucrose, 0.038 M KH₂PO₄, 0.072 M K₂HPO₄ at pH 7.1]) was added to a final concentration of 1× concentration, vortexed, aliquoted, and flash-frozen in a dry ice/ethanol bath for storage at −70° C.

ii. Plasmids

Two subclones spanning the areas of interest were utilized to make nucleotide changes in the full length rA2ΔM2-2(MEDI) cDNA. The subclones were derived from plasmid pA2ΔM2-2 described previously (Jin et al., 2000, J. Virol. 74:74-82). The first subclone was generated by digesting pA2ΔM2-2 with KpnI and XhoI, and ligating the 4482 bp fragment into plasmid pCITE-2a. The resulting clone was designated pCITERSV/K-X, and includes nucleotides (nt) 1 to 4482 of the rA2ΔM2-2(MEDI) cDNA. The second subclone was generated by digesting plasmid pA2ΔM2-2 with XhoI and BamHI and ligating the 3785 bp fragment into plasmid pCR-2.1. The resulting subclone was designated pCR2.1RSVΔM2-2/X-B and includes nt 4482-8267 of the rA2ΔM2-2(MEDI) genome. Nucleotide changes in each subclone were made using Quickchange site-directed mutagenesis per manufacturer's instructions (Agilent). Nucleotide changes were confirmed by sequencing, and the fragments were inserted back into the full-length pA2ΔM2-2 cDNA using the same paired restriction enzymes described above for each subclone. For transfection experiments requiring expression of the full-length RSVA2 F protein, the 1725 nucleotide sequence of the RSV F ORF was codon optimized at Medimmune and synthesized by DNA2.0. The ORF was amplified by PCR and cloned into plasmid pCMV-Script (Agilent). This plasmid was designated pCMV/RSVF. Nucleotide changes in the RSV F sequence were made using Quickchange site-directed mutagenesis (Agilent).

iii. Rescue of Recombinant rRSVA2ΔM2-2 Virus

6-well plates of sub-confluent BSR/T7 cells were co-transfected with plasmid encoding the full length cDNA as well as helper plasmids encoding the RSV A2 N, P, M2-1 and L genes under the control of the T7 promoter. Briefly, 4 μg of full-length cDNA was mixed with 0.4 μg pCITE/RSV N, 0.4 μg pCITE/RSV P, 0.3 μg pCITE/RSV L and 0.2 μg pCITE/RSV M2-1, and 8 μL Lipofectamine2000 (Invitrogen) in a final volume of 0.2 mL Optimem-I. BSRT7 cells were washed and 0.5 mL of Optimem-I was added followed by 0.2 mL of transfection mix. Plates were incubated overnight at 35° C. The following day the transfection mix was removed and replaced with 2 mL of Optimem-I. After 5 days incubation at 35° C. in a 5% CO₂ incubator cells and supernatant were harvested together, and any rescued virus was amplified by 2-3 passages in Vero cells. Viral titers were determined by plaque assay.

The sequence of each recovered virus was confirmed by RT-PCR. Briefly, the viral RNA was isolated using a Qiamp viral RNA minikit (Qiagen). RT-PCR was performed using a OneStep RT-PCR kit (Qiagen) and oligonucleotide primers that generated overlapping PCR products to cover the entire genome. Gel extracted PCR products (Qiagen) were sent to Sequetech Inc for sequencing.

iv. Plaque Assay

Virus titers were determined by plaque assay in Vero cells. Briefly, virus stocks were serially diluted and 0.5 mL of each dilution was used to infect one well of a 6-well plate containing sub-confluent Vero cells. After 1 hour rocking at room temperature, virus was aspirated and wells were overlayed with a 1:1 mixture of 2% methylcellulose and 2XL-15/EMEM (SAFC) medium supplemented with 2% heat-inactivated FBS, 4 mM L-glutamine, and 200 U penicillin with 200 ug/mL streptomycin. Plates were incubated at 35° C. in a 5% CO₂ incubator. Following 5-6 days incubation, the overlay was removed by aspiration, plates were fixed in methanol, and the fixed cells were immunostained using polyclonal anti-RSV antibody (Millipore) diluted 1:1000 in 5% powdered milk (w/v) in phosphate buffered saline (PBS), followed by horse radish peroxidase (HRP)-conjugated rabbit antibody (Ab) directed to goat Ab (Dako). Plaques were visualized with 3-amino-9-ethylcarbazole (Dako). Virus titer is reported as plaque forming units (PFU)/ml.

vi. Multi-Cycle Growth Analysis of Recombinant rRSVA2ΔM2-2 Virus

6-well plates of subconfluent Vero cells were infected at a multiplicity of infection (MOI) of 0.1 PFU/cell in 0.5 mL of Optimem-I per well. Plates were rocked at room temperature for 1 hour to facilitate virus absorption and washed once with Optimem-I, followed by addition of 2 mL fresh medium Plates were incubated at 35° C. in a 5% CO₂ incubator and virus was harvested at the indicated time points and prepared for −70° C. storage as described. Virus titers were determined by plaque assay as described.

vii. Syncytium Formation Assay

Sub-confluent Vero cells in 6-well plates were transfected overnight with 1 μg per well of plasmid pCMV/RSVF or its derivatives. Briefly, transfection mix was generated by mixing 4 μL of Lipofectamine2000 (Life Technologies) per 1 μg of plasmid DNA in a final volume of 0.2 mL Optimem-I. Cells were washed once and 0.5 mL of Optimem-I was added, followed by 0.2 mL of transfection mix per well. After overnight incubation at 37° C. in a 5% CO₂ incubator, plates were washed and 2 mL per well Optimem-I was added before returning to 37° C. incubation. Syncytium formation was examined at various time points post-transfection, and images were captured using a Nikon Eclipse TS100 microscope.

viii. Western Blotting

6-well plates of Vero cells were transfected as described above. At 48 hours post-transfection, cell lysates were harvested by aspirating the medium, washing the well with PBS, and adding 0.3 mL Laemmli buffer+β-mercaptoethanol directly to each well. Before loading onto 12% Tris-glycine SDS-PAGE gels, lysates were incubated at 95° C. for 10 min. Gels were blotted to polyvinylidene difluoride (PVDF) membrane (Invitrogen) and probed with motavizumab diluted to 0.1 μg/mL in 5% milk in PBS followed by HRP-conjugated anti-human secondary antibody (Dako). β-actin was detected with a monoclonal antibody directed against chicken actin (Millipore) followed by HRP-conjugated anti-mousesecondary antibody (Dako). Electrochemiluminescence (ECL) was developed using Supersignal Dura West ECL substrate (Pierce) and visualized on ImageQuant LAS4000 imager.

ix. Immunofluorescence

Vero cells were seeded to 90% confluency in 12-well plates containing sterile glass coverslips. Transfections were performed as described above but scaled for 12-well plates. At 48 hours post-transfection cells were fixed with 4% paraformaldehyde in PBS for 20 min at room temperature. Plates were blocked with PBS+1% BSA for 1 h at 37° C. and incubated with primary antibody (0.5 μg/mL motavizumab in PBS+1% BSA+0.1% saponin) for 1 h at 37° C. Plates were washed with PBS-Tween followed by addition of secondary antibody (AlexaFluor 488 goat anti-human IgG, 4 μg/mL in PBS+1% BSA+0.1% saponin). After 1 hour at 37° C., plates were washed with PBS-Tween. Coverslips were inverted and mounted on glass slides using Vectashield mounting medium with DAPI (Vector Labs). Images were captured at 10× magnification using a Nikon Eclipse 80i microscope with CoolSnapES2 camera and Simple PCI6 software.

x. Flow Cytometry

To assess cell surface expression of RSV F, 293T cells were transfected as described above. At 20 hours post-transfection cells were stained for FACS analysis using motavizumab followed by Alexafluor488 anti-human antibody, each at a concentration of 1 μg/mL. Cells were analyzed on LSR-II and mean fluorescence intensity (MFI) was determined using FACSDiva software.

What is claimed is:

1. A recombinant respiratory syncytial virus (RSV) having an attenuated phenotype in which a naturally occurring amino acid found at position 66 of an F subunit shown in SEQ ID NO:2 is substituted with an amino acid residue having a positively charged side chain.

2. A recombinant respiratory syncytial virus (RSV) having an attenuated phenotype, the RSV comprising an F subunit having an amino acid sequence that it at least about 95% identical to an amino acid sequence shown in SEQ ID NO: 2, wherein a naturally occurring amino acid found at position 66 of an amino acid sequence shown in SEQ ID NO:2 is substituted with an amino acid residue having a positively charged side chain.

3. The recombinant RSV according to claim 1 or 2, wherein the naturally occurring amino acid at position 66 is substituted with an amino acid selected from the group consisting of arginine (R), lysine (K) and histidine (H).

4. The recombinant RSV according to any of claims 1-3, further comprising an M2-2 protein comprising a mutation that renders the M2-2 protein inactive.

5. The recombinant RSV according to any of claims 1-3, further comprising an M2-2 protein comprising a mutation that prevents expression of the M2-2 protein.

6. The recombinant RSV according to claim 4 or 5, wherein the M2-2 protein comprises an amino acid sequence that is at least 95% identical to SEQ ID NO:4.

7. The recombinant RSV according to claims 4 to 6, wherein the M2-2 protein comprises a deletion mutant in which at least about 5 amino acid residues are deleted from an amino acid sequence shown in SEQ ID NO:4.

8. The recombinant RSV according to claims 4 to 6, comprising a M2-2 deletion mutant in which at least about 5% amino acids are deleted from an amino acid sequence shown in SEQ ID NO: 4.

9. The recombinant RSV according to claim 7 or 8, wherein one or more amino acids are deleted from an N-terminus of the amino acid sequence shown in SEQ ID NO:4.

10. The recombinant RSV according to claim 7 or 8, wherein one or more amino acids are deleted from a C-terminus of the amino acid sequence shown in SEQ ID NO:4.

11. An isolated nucleic acid encoding a recombinant respiratory syncytial virus (RSV) having an attenuated phenotype, the isolated nucleic acid encoding an F subunit in which a naturally occurring amino acid found at position 66 of SEQ ID NO:2 is substituted with an amino acid residue having a positively charged side chain.

12. An isolated nucleic acid encoding a recombinant respiratory syncytial virus (RSV) having an attenuated phenotype comprising a nucleic acid sequence that it at least about 95% identical to a nucleic acid sequence shown in SEQ ID NO: 1, wherein the isolated nucleic acid encodes an F subunit in which a naturally occurring amino acid found at position 66 of SEQ ID NO:2 is substituted with an amino acid residue having a positively charged side chain.

13. The isolated nucleic acid according to claim 11 or 12, wherein the naturally occurring amino acid at position 66 is substituted with an amino acid selected from the group consisting of arginine (R), lysine (K) and histidine (H).

14. The isolated nucleic acid according to any one of claims 11-13, wherein the isolated nucleic acid encodes recombinant RSV comprising an M2-2 protein comprising a mutation that renders the M2-2 protein inactive.

15. The isolated nucleic acid according to any one of claims 11-13, wherein the isolated nucleic acid encodes recombinant RSV comprising an M2-2 protein comprising a mutation that prevents expression of the M2-2 protein.

16. The isolated nucleic acid according to claim 14 or 15, wherein the isolated nucleic acid encodes recombinant RSV comprising a nucleic acid sequence that is at least 95% identical to SEQ ID NO: 3.

17. The isolated nucleic acid according to claims 14 to 16, wherein the isolated nucleic acid encodes recombinant RSV comprising a M2-2 deletion mutant in which at least about 5 amino acid residues are deleted from an amino acid sequence shown in SEQ ID NO:4.

18. The isolated nucleic acid according to claims 14 to 15, wherein the isolated nucleic acid encodes recombinant RSV comprising a M2-2 deletion mutant in which at least about 5% amino acids are deleted from an amino acid sequence shown in SEQ ID NO:4.

19. The isolated nucleic according to claim 17 or 18, wherein one or more amino acids are deleted from an N-terminus of the amino acid sequence shown in SEQ ID NO:4.

20. The isolated nucleic acid according to claim 17 or 18, wherein one or more amino acids are deleted from a C-terminus of the amino acid sequence shown in SEQ ID NO:4.

21. The isolated nucleic acid according to any one of claims 11-20, wherein the nucleic acid comprises DNA or RNA.

22. The isolated nucleic acid according to any one of claims 11-21, wherein the nucleic acid comprises mRNA.

23. A vector comprising the nucleic acid according to any one of claims 11-22.

24. A vector comprising a sequence at least 95% identical to SEQ ID NO: 6.

25. A respiratory syncytial virus (RSV) vaccine comprising an immunologically effective amount of a recombinant RSV, wherein recombinant RSV has an attenuated phenotype in which a naturally occurring amino acid found at position 66 of SEQ ID NO:2 is substituted with an amino acid residue having a positively charged side chain.

26. A respiratory syncytial virus (RSV) vaccine comprising an immunologically effective amount of a recombinant respiratory syncytial virus (RSV) having an attenuated phenotype, the RSV comprising an F subunit having an amino acid sequence that it at least about 95% identical to an amino acid sequence shown in SEQ ID NO: 2, wherein a naturally occurring amino acid found at position 66 of an amino acid sequence shown in SEQ ID NO:2 is substituted with an amino acid residue having a positively charged side chain.

27. The RSV vaccine according to claim 25 or 26, wherein the naturally occurring amino acid at position 66 is substituted with an amino acid selected from the group consisting of arginine (R), lysine (K) and histidine (H).

28. The RSV vaccine according to any one of claims 25-27, wherein recombinant RSV further comprises an M2-2 protein comprising a mutation that renders the M2-2 protein inactive.

29. The RSV vaccine according to any one of claims 25-27, wherein recombinant RSV further comprises an M2-2 protein comprising a mutation that prevents expression of the M2-2 protein.

30. The RSV vaccine according to claim 28 or 29, wherein the M2-2 protein comprises an amino acid sequence that is at least 95% identical to SEQ ID NO:4.

31. The RSV vaccine according to claims 28 to 30, wherein recombinant RSV comprises a M2-2 deletion mutant in which at least about 5 amino acid residues are deleted from an amino acid sequence shown in SEQ ID NO:4.

32. The RSV vaccine according to claims 28 to 30, wherein recombinant RSV comprises a M2-2 deletion mutant in which at least about 5% of amino acids are deleted from an amino acid sequence shown in SEQ ID NO:4.

33. The RSV vaccine according to claim 31 or 32, wherein one or more amino acids are deleted from an N-terminus of the amino acid sequence shown in SEQ ID NO:4.

34. The RSV vaccine according to claim 31 or 32, wherein one or more amino acids are deleted from a C-terminus of the amino acid sequence shown in SEQ ID NO:4.

35. The RSV vaccine according to any one of claims 25-34, further comprising a pharmaceutically acceptable carrier and/or adjuvant.

36. A pharmaceutical composition comprising recombinant RSV according to any one of claims 1-10.

37. A method of stimulating a protective immune response against respiratory syncytial virus (RSV), the method comprising administering an immunologically effective amount of recombinant RSV having an attenuated phenotype according to any one of claims 1-10 to a mammal.

38. A method of preventing disease caused by respiratory syncytial virus (RSV), the method comprising administering an immunologically effective amount of recombinant RSV having an attenuated phenotype according to any one of claims 1-10 to a mammal.

39. A method of inducing neutralizing antibodies against respiratory syncytial virus (RSV) in a mammal, the method comprising administering an immunologically effective amount of recombinant RSV having an attenuated phenotype according to any one of claims 1-10 to a mammal.

40. A method of reducing respiratory syncytial virus (RSV) viral titres, the method comprising administering an immunologically effective amount of recombinant RSV having an attenuated phenotype according to any one of claims 1-10 to a mammal.

41. The method according to any one of claims 37-40, wherein the mammal is human.

42. The method according to any one of claims 37-41, wherein recombinant RSV is administered in a single dose.

43. The method according to any one of claims 37-41, wherein recombinant RSV is administered in more than one dose.
